(19) United States
(12) Patent Application Publication
(10) Pub. No.: US 2026/0109739 A1
(43) Pub. Date: Apr. 23, 2026

(54) IN VIVO IMMOBILIZATION OF PROTEINS

(71) Applicant: The Chinese University of Hong Kong, Shatin (CN)

(72) Inventors: Michael CHAN, Kowloon (CN); Bradley Heater, Shatin (CN); Marianne Lee, Kowloon (CN)

(21) Appl. No.: 19/300,559

(22) Filed: Aug. 14, 2025

Related U.S. Application Data
(62) Division of application No. 17/605,993, now Pat. No. 12,404,308.
(60) Provisional application No. 62/839,400, filed on Apr. 26, 2019.

(51) Int. Cl. C07K 14/325; C12N 15/70; C12N 15/75

(52) U.S. Cl. CPC C07K 14/325; C12N 15/70; C12N 15/75; C07K 2319/00

(57) ABSTRACT

The present invention provides a novel system for immobilizing recombinantly produced proteins by entrapping them in crystals of co-expressed proteins that are capable of self-crystallization. Related compositions and as well as methods of making and using the immobilized proteins are also described.

### RELATED APPLICATIONS

This application is a divisional of U.S. patent application Ser. No. 17/605,993, filed Oct. 22, 2021, which is a 371 National Stage Entry of PCT Patent Application No. PCT/CN2020/086652, filed Apr. 24, 2020, which claims priority to U.S. Provisional Patent Application No. 62/839,400, filed Apr. 26, 2019, the contents of which are hereby incorporated by reference in the entirety for all purposes.

### REFERENCE TO SUBMISSION OF A SEQUENCE LISTING AS A TEXT FILE

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Nov. 20, 2025, is entitled “080015-1516301-027720US_ST26.xml”, and is 39,321 bytes in size.

### BACKGROUND OF THE INVENTION

In many medical and industrial contexts the use of recombinant proteins is becoming increasingly more important. The production, isolation, use, and potential reuse of recombinant proteins, especially enzymes, remain in need of improvement for better quality, efficiency, and stability. For instance, enzyme immobilization can facilitate easy removal and subsequent reuse of enzymes during multiple rounds of catalysis. In many cases, immobilization also improves the stability of enzymes against many industrial conditions such as high temperatures and organic solvents. Immobilization by entrapment is particularly attractive since it does not involve any modifications to the enzyme structure, increasing the chance for high activity retention and native enzyme conformation. Thus, there exists a need for new and effective means for producing recombinant proteins, such as enzymes, in immobilized form. The present invention fulfills this and other related needs.

### BRIEF SUMMARY OF THE INVENTION

This invention provides a novel approach to improve the physical properties and stability of recombinant proteins by immobilizing recombinant proteins such as enzymes useful in various industrial applications. Thus, in a first aspect, this invention provides a method for recombinantly co-expressing a protein of interest with a crystal-forming protein. The method includes these steps: (1) providing bacterial cells comprising an expression cassette encoding the protein of interest and an expression cassette encoding a Cry protein, a crystal-forming fragment of the Cry protein, or a fusion protein capable of forming crystals comprising the Cry protein or the crystal-forming fragment thereof, and (2) culturing the bacterial cells under conditions permissible for the expression of the protein of interest as well as the Cry protein, the crystal-forming fragment thereof, or the fusion protein, wherein the Cry protein, the crystal-forming fragment thereof, or the fusion protein forms crystal containing the protein of interest upon both being expressed in the bacterial cells.

In some embodiments, the protein of interest is an enzyme, such as a lipase (e.g., Proteus mirabilis lipase, or PML, including a PML variant with modifications of residues 118 and 130, for example, I118V+E130G, and lipA or lipAR9), ligase, hydrolase, esterase, protease, or glycosidase. In some embodiments, the Cry protein is Cry3Aa. In some embodiments, the crystal-forming fragment is the N-terminal 290 amino acids of Cry3Aa, or the N-terminal 625 or 626 amino acids of Cry3Aa, or the 498-644 fragment of Cry3Aa. In some embodiments, the expression cassette encoding the protein of interest and the expression cassette encoding the Cry protein, crystal-forming fragment thereof, or the fusion protein is one and the same expression cassette. In some embodiments, the one single expression cassette comprises (1) one copy of polynucleotide sequence encoding the Cry protein, crystal-forming fragment thereof, or the fusion protein, and (2) one copy or two or more copies of polynucleotide sequence encoding the protein of interest. In some embodiments, (1) the polynucleotide sequence encoding the Cry protein, crystal-forming fragment thereof, or the fusion protein, and (2) the polynucleotide sequence encoding the protein of interest are operably linked to one single promoter. In some embodiments, the one single promoter is operably linked to (1) one copy of the polynucleotide sequence encoding the Cry protein, crystal-forming fragment thereof, or the fusion protein, followed by (2) one copy of the polynucleotide sequence encoding the protein of interest, with one ribosome binding site between (1) and (2). In some embodiments, the one single promoter is operably linked to (1) one copy of the polynucleotide sequence encoding the Cry protein, crystal-forming fragment thereof, or the fusion protein, followed by (2) two or more copies of the polynucleotide sequence encoding the protein of interest, with one ribosome binding site between (1) and (2) and between two copies of polynucleotide sequence encoding the protein of interest.

In some embodiments, (1) the polynucleotide sequence encoding the Cry protein, crystal-forming fragment thereof, or the fusion protein; and (2) the polynucleotide sequence encoding the protein of interest are operably linked to two separate promoters. In some embodiments, the two separate promoters are two different kinds of promoters, for example, cyt1Aa promoter and cry3Aa promoter. In some embodiments, (1) the polynucleotide sequence encoding the Cry protein, crystal-forming fragment thereof, or the fusion protein; and (2) the polynucleotide sequence encoding the protein of interest share one single termination codon, resulting in one copy of the Cry protein, crystal-forming fragment thereof, or the fusion protein and two copies of the protein of interest.

In some embodiments, the expression cassette encoding the protein of interest and the expression cassette encoding the Cry protein, crystal-forming fragment thereof, or the fusion protein are two separate expression cassettes. In some embodiments, the fusion protein comprises the Cry protein or crystal-forming fragment thereof and one or more heterologous polypeptides (such as a lipase lipA or SEQ ID NO:8 in 1-3 repeats) at the N- and/or C-terminus. In some embodiments, the fusion protein is Cry3Aa-[SmtA]₁₋₃. In some embodiments, two or more proteins of interest are co-expressed with the Cry protein, the crystal-forming fragment thereof, or the fusion protein and are contained within the crystal formed by the Cry protein, the crystal-forming fragment thereof, or the fusion protein. In some embodiments, the bacterial cells are Bacillus subtilis (Bs) or Bacillus thuringiensis (Bt) cell or E. coli cells. In some embodiments, the method of this invention further includes a step, prior to step (1), of introducing into the bacterial cells the expression cassette encoding the protein of interest and the expression cassette encoding the Cry protein, crystal-forming fragment thereof, or the fusion protein. In some embodiments, more than one protein of interest, e.g., two or more proteins, are recombinantly co-expressed with the Cry protein, crystal-forming fragment thereof, or the fusion protein. These proteins of interest may be the same protein (e.g., both are PML) or different proteins (e.g., one lipase and one ligase). In some embodiments, the method of this invention further includes a step, after step (2), of isolating the crystal formed by the Cry protein, the crystal-forming fragment thereof, or the fusion protein and entrapping the protein or proteins of interest, which may be more than one protein, e.g., two or more proteins. In some embodiments, after it is isolated, the crystal containing the protein(s) of interest is washed under appropriate conditions such as choosing appropriate salt concentration etc. to permit the protein(s) entrapped within the crystal to be released from the crystal, preferably without dissolving the crystal to any substantial degree. In some embodiments, after being isolated, the crystal containing the protein(s) of interest is dissolved to release the protein(s) entrapped within the crystal. In some embodiments, the protein is an enzyme, such as PML. In some embodiments, the protein of interest is a fluorescent protein such as mCherry.

In a second aspect, the present invention provides a protein crystal produced by the method described above and herein for co-expression of one or more recombinant proteins of interest with a crystal-forming protein, such as a Cry protein, a crystal-forming fragment of the Cry protein, or a fusion protein capable of forming crystals comprising the Cry protein or the crystal-forming fragment thereof. In some embodiments, the protein of interest is an enzyme, such as a lipase (e.g., Proteus mirabilis lipase, or PML, including a PML variant with modifications of residues 118 and 130, for example, I118V+E130G, and lipA or lipAR9), ligase, hydrolase, esterase, protease, or glycosidase. In some embodiments, the protein of interest is a fluorescent protein such as mCherry.

In a third aspect, the present invention provides a method for performing a reaction. The method comprises the step of incubating the protein crystal produced by the method of this invention entrapping an enzyme therein with a substrate to the enzyme under conditions permissible for the substrate to be catalyzed by the enzyme. In some embodiments, the enzyme is a lipase (e.g., Proteus mirabilis lipase, or PML, including a PML variant with modifications of residues 118 and 130, for example, I118V+E130G, and lipA or lipAR9), ligase, hydrolase, esterase, protease, or glycosidase. In some embodiments, the method further comprises a step, after the reaction is completed, of removing the reaction product and cleaning, e.g., washing the crystal to remove any detectable amount of reaction mixture including reaction agents and/or product(s), preferrably without dissolving the crystal to any substantial degree, and then reusing the protein crystal in a second reaction.

In a fourth aspect, the present invention provides an in vitro method for co-crystallizing (1) a Cry protein, a crystal-forming fragment of the Cry protein, or a fusion protein capable of forming crystals comprising the Cry protein or the crystal-forming fragment thereof with (2) one or more proteins (e.g., enzymes) by mixing a soluble protein described in (1) with the protein or proteins of (2), thus allowing the protein or protein(s) of (2) to be entrapped within a protein crystal formed by the crystal-forming protein of (1). In some embodiments, the protein is an enzyme. In some embodiments, the enzyme-entrapped protein crystal so formed is used for performing a reaction where the protein crystal is incubated with a substrate to the enzyme under conditions permissible for the substrate to be catalyzed by the enzyme. In some embodiments, the protein crystal containing entrapped protein(s) of interest formed by the methods described above and herein is used for delivering the protein(s) to cells, such as macrophages, lymphocytes, cancer cells, red blood cells, epithelial cells, stem cells, and liver cells.

In a fifth aspect, the present invention provides various modified Cry proteins, their fragments or fusion proteins, all of which still retaining the crystalizing capability (for example, Cry3Aa*, Cry3Aa*-lipA, Cry3Aa*-SpyCat, Cry3Aa-[SmtA]₁₋₃, NegCry3Aa, 3A2-2, Cry3Aa (S145C, H161R), as well as various modified proteins with retained enzymatic activities (such as PML, PML^{VG}, LmSP, LipA, and LipAR9), a polynucleotide sequence encoding each of such proteins, a nucleic acid comprising the polynucleotide sequence encoding each of the proteins, especially an expression cassette comprising the polynucleotide coding sequence operably linked to a promoter (e.g., a heterologous promoter from an origin different from that of the wild-type base protein) or a vector comprising such an expression cassette, a host cell comprising such a vector or expression cassette, which is able to express the protein under permissible culture conditions. Also provided are methods for recombinantly producing any of these proteins by culturing the host cells under conditions permissible for the recombinant protein expression and optionally further concentrating or isolating/purifying the proteins.

### BRIEF DESCRIPTION OF THE DRAWINGS

(FIG. 1A) Electron micrograph of Bt. highlighting its crystal inclusion. Electron micrographs of purified (FIG. 1B) Cry3Aa crystals, and (FIG. 1C) (Cry3Aa-GFP fusion crystals (reproduced from reference 15).

FIG. 2. Recyclability of Cry3Aa*-lipA for the conversion of coconut oil to biodiesel.¹⁶

FIG. 3. Schematic illustrating the potential mode of entrapment of PML within Cry3Aa crystals (modified from reference 16).

FIG. 4. Plasmid variations constructed for optimization of PML entrapment in Cry3Aa crystals. 3P-cry3Aa promoter; RBS-ribosome binding site.

FIG. 5A.-FIG. 5B SDS-PAGE analysis of PML^{VG} entrapped in Cry3Aa crystals by different coexpression methods. (FIG. 5A) Analysis of Cry3Aa[PML^{VG}] and 3PPML^{VG}[Cyt13A] crystals and (FIG. 5B) Cry3Aa[PML^{VG}]² and 3P3A[3PPML^{VG}] crystals. Lane (M) molecular weight marker (kDa).

FIG. 6. Scanning electron micrographs of Cry3Aa and 3P3A3P[PML^{VG}] crystals.

FIG. 7. p-Nitrophenyl palmitate (pNPP) hydrolysis activity of PML^{VG} entrapped in Cry3Aa crystals by different coexpression methods.

FIG. 8A. Recyclability of optimized Cry3Aa[PML^{VG}] crystals during the conversion of waste cooking oil to biodiesel. Cycles were 10 h each. FIG. 8B. Thermal stability of free PML^{VG} and 3P3A[3PPML^{VG}] crystals. Samples were incubated in triplicate at various temperatures for 1 h and then tested for residual activity. Error bars show the standard deviation of the mean.

FIG. 9A-FIG. 9C GCMS analysis of benzyl laurate synthesis reaction. (FIG. 9A) Elution profile of no enzyme control shows no benzyl laurate peak. (FIG. 9B) Elution profile of lipase crystals reaction to produce benzyl laurate. (FIG. 9C) Mass-spec of benzyl laurate peak confirms product formation.

FIG. 10. In vitro co-crystallization of Cry3Aa and carboxy rhodamine labelled PML by vapor diffusion. Crystals with red fluorescence but no UV fluorescence are crystals comprised of PML only. Crystals with UV fluorescence but not red fluorescence are Cry3Aa crystals that do not contain PML. Entrapment of PML inside Cry3Aa crystals is confirmed by finding crystals with both red and UV fluorescence.

FIG. 11. Fluorescence microscopy of Cry3Aa co-entrapment of GFP and mCherry fluorescent proteins. The data demonstrate that GFP and mCherry proteins are entrapped in single Cry3Aa crystals.

FIG. 12A-FIG. 12C Production and analysis of LmSP entrapment in Cry3Aa crystals. (FIG. 12A) SDS-PAGE analysis of 3P3A3P[LmSP] and 3P3A3P[LmSP][PML^{VG}] crystals. (FIG. 12B) Sucrose phosphorylase activity and (FIG. 12C) lipase activity of coentrapped crystals.

FIG. 13A-FIG. 13B Production and characterization of Cry3Aa*[PML^{VG}]² crystals. (FIG. 13A) SDS-PAGE gel of purified Cry3Aa*[PML^{VG}]2 crystals. Lane (1) molecular weight marker (kDa), Lane (2) crystals. (FIG. 13B) pNPP hydrolysis activity of Cry3Aa*[PML^{VG}]2 crystals.

FIG. 14A-FIG. 14B. Production and activity of the lipase:lacasse catalyst. (FIG. 14A) SDS-PAGE gel showing conjugation of CotA-Spytag to Cry3Aa*-SpyCat[PML^{VG}] crystals at different ratios. Lane (M) molecular weight marker (kDa). (FIG. 14B) 2,2′-azino-bis(3-ethylbenzothiazoline-6-sulphonic acid) (ABTS) oxidation by free CotA-Spytag and Cry3Aa*-SpyCat[PML^{VG}] crystals.

FIG. 15A-FIG. 15B Production and characterization of Cry3Aa*-lipA[PML^{VG}] crystals. (FIG. 15A) SDS-PAGE analysis of purified Cry3Aa*-lipA[PML^{VG}] crystals. Lane (1) Pure crystals; lane (M) molecular weight marker (kDa). (FIG. 15B) Time-point study of waste cooking oil conversion to biodiesel.

FIG. 16A. Scheme 1: Lipase catalyzed tranesterification of triacylglycerols with methanol (MeOH) to produce biodiesel. Scheme 2: Lipase catalyzed esterification of benzyl alcohol and lauric acid in neat isooctane to produce the cosmetic compound benzyl laurate. FIG. 16B. Scheme 3: Reaction scheme for the one-pot synthesis of biodiesel and αGG using PML lipase and LmSP sucrose phosphorylase. Scheme 4: Reactions scheme for one-pot production of azelaic acid using PML lipase and CotA-lacasse.

FIG. 17. Amino acid sequence alignment between Cry3Aa (SEQ ID NO:4) and NegCry3Aa (SEQ ID NO:6).

FIG. 18A-FIG. 18B Purified negCry3Aa-RBS-scGFP protein crystals. (FIG. 18A) NegCry3Aa-RBS-scGFP crystal pellets purified from Bt cells grown for 48 h (left) and 72 h (right) were imaged using BioRad ChemiDoc MP System at the GFP excitation wavelength (ex=488 nm). (FIG. 18B) SDS-PAGE gel showing the corresponding purified NegCry3Aa-RBS-scGFP crystals in (A).

FIG. 19A-FIG. 19B Entrapment of lipAR9 in negCry3Aa crystals. (FIG. 19A) SDS-PAGE analysis of Cry3Aa[lipAR9] and negCry3Aa[lipAR9] crystals. (FIG. 19B) Quantitation of loading of lipAR9 in Cry3Aa and negCry3Aa crystals.

FIG. 20. SDS-PAGE of Cry3Aa-[SmtA]1 produced in Bt and purified by sucrose density gradient centrifugation. Lane 1: BioRad Precision plus molecular weight marker; lane 2-3: Extracts from <40% sucrose layers; lane 4: crystal extracts from the interface of 40%/60% sucrose layer.

FIG. 21 Structure of Cry3Aa crystal channel. Residues highlighted in red are regions exposed to the solvent channel and can be deleted to expand the channel size.

FIG. 22 Cry3Aa protein sequence. Residues in bold are regions exposed to the solvent channel and can be deleted to expand the channel size. (SEQ ID NO: 4).

FIG. 23. Amino acids in specific regions exposed to the Cry3Aa (SEQ ID NO:4) crystal channel.

FIG. 24A-FIG. 24B Entrapment of LmSP in 3A2-2 crystal. (FIG. 24A) Model of LmSP homolog Thermoanaerobacter thermosaccharolyticum 6F-phosphate phosphorylase (PDB ID: 6S9V) inside the Cry3Aa crystal (PDB ID: 1DLC) channel. Loop regions that are oriented in the channel with potential steric clash with LmSP are colored red. LmSP is colored yellow with its amino acids colored in black and portrayed as lines. Cry3Aa is colored blue. Model was produced in COOT and images were prepared in PyMol. (FIG. 24B) SDS-PAGE analysis after entrapment of LmSP in the wild-type Cry3Aa crystal (3A[LmSP]) and in the mutant 3A2-2 crystal (3A2-2[LmSP]).

FIG. 25A-FIG. 25D Stabilizing mutations in Cry3Aa protein. (FIG. 25A) Structure of two Cry3Aa monomers showing His161-E168 electrostatic interaction. (FIG. 25B) Solubilization resistance of Cry3Aa mutants. (FIG. 25C) Structure of two Cry3Aa monomers showing potential site for disulfide formation. (FIG. 25D) Recyclability of PML^{VG} triple copy constructs 3A[PML^{VG}]³ and double mutant 3ADM[PML^{VG}]³ during the synthesis of biodiesel from waste cooking oil.

FIG. 26A-FIG. 26B Catch and release of lipAR9 by Cry3Aa crystals. (FIG. 26A) p-Nitrophenyl acetate hydrolysis activity of purified 3A[lipAR9] crystals. (FIG. 26B) Lipase activity of supernatant after release of lipAR9 protein. 3A[lipAR9] crystals were incubated with increasing concentrations of NaCl for 1 h and then removed from the mixture. The increase in lipase activity in the supernatant indicates that lipA was released as a function of NaCl concentration.

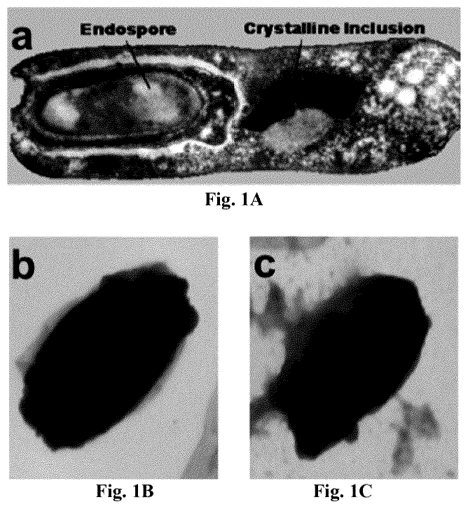

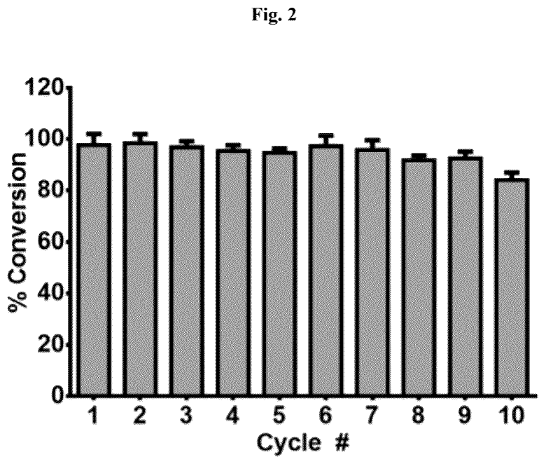

### DEFINITIONS

The term “Cry protein,” as used herein, refers to any one protein among a class of crystalline proteins produced by strains of Bacillus thuringiensis (Bt). Some examples of “Cry proteins” include, but are not limited to, Cry1Aa, Cry1Ab Cry2Aa, Cry3Aa, Cry4Aa, Cry4Ba, Cry11Aa, Cry11Ba, and Cry19Aa. Their amino acid sequences and polynucleotide coding sequences are known and can be found in publications such as U.S. patent application published as US2010/0322977. Their GenBank Accession Nos are:
Cry1Aa AY197341.1
Cry1Ab AY847289.1
Cry2Aa AF273218.1
Cry3Aa AJ237900.1
Cry4Aa AB513706.1
Cry4Ba AB161456.1
Cry11Aa AL731825.1
Cry11Ba LC153032.1
Cry19Aa Y07603.1

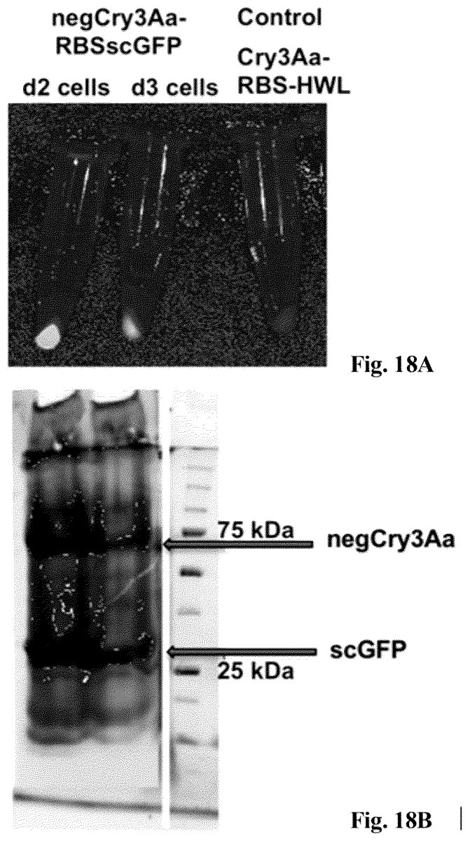

In addition to the wild-type Cry proteins, the term “Cry protein” also encompasses functional variants, which (1) share an amino acid sequence identity of at least 80%, 81%, 82%, 83%, 84%, 88%, 89%, 90%, 91%, 92%, 93%, 94%, 95%, 96%, 97%, 98%, 99%, or 99.5% to the polypeptide sequence of any one of the Cry proteins listed in US2010/0322977; and (2) retain the ability to spontaneously form crystals within host cells as can be confirmed by known methods such as electron micrograph (see description in, e.g., Park et al., Appl Environ Microbiol, 1998, 64, 3932-3938; Schnepf et al., Microbiol Mol Biol Rev, 1998, 62, 775-806; Whiteley and Schnepf, Annu Rev Microbiol, 1986, 40, 549-576; and Nair et al., PLoS One, 2015, 10, e0127669). For example, a “Cry protein” encompasses any variant that confers increased negative charges to the resultant protein by substitutions of at least 3, 4, 5, 6, 7, 8, 9, 10, 11, 12, 13, 14, 15, or more positively charged or neutral amino acids within the domain II of the wild-type Cry protein (e.g., the 295 to 499 segment of SEQ ID NO:4, as well as its corresponding segment in other Cry proteins as shown in FIG. 27) with negatively charged amino acids such as aspartate (D) and glutamate (E). One example of such mutant is NegCry3Aa, SEQ ID NO:6, which is a Cry3Aa variant containing the following mutations: K384E, N391D, N395D, S425E, Q430E, TQ436437EE, KR442443EE, T461D, and K467E. Further encompassed by the term “Cry protein” are modified Cry proteins containing amino acid insertions or deletions (for example, insertion of at least 1, 2, 3, 4, 5, 6, 7, 8, 9, or 10 amino acids, or a deletion of at least 1, 2, 3, 4, 5, 6, 7, 8, 9, 10, or more amino acids) at one or more of the 8 specific regions (for example, at least 1, 2, 3, 4, 5, 6, 7, or all 8 regions) shown in FIG. 22, as well as their corresponding regions in other Cry proteins as shown in FIG. 18 of PCT/CN2020/084939, can effectively reduce or enlarge the protein's channel size, respectively, and can therefore make the resultant crystal-forming protein variant to more effectively entrap a target protein (such as an enzyme) with increased retention rate and reduced diffusion rate for the target protein. Typically, such a channel-size altering Cry protein mutant contains at least 3, 4, or 5 but no more than 10, 12, or 15 point mutations (deletions or insertions) in the entire protein. One example of a Cry3Aa mutant of this nature is termed 3A2-2 (SEQ ID NO:7). In addition, the term “Cry protein” also encompasses Cry protein mutants that have been modified at one or more residues to improve the stability of the resultant protein or its crystal structure, for example, introducing one or more cysteine residues by insertion or substitution to the wild-type Cry protein amino acid sequence so as to form additional disulfide bond or bonds to stabilize the protein or crystal structure. One such example is a double mutant of Cry3Aa (3AS145C, H161R), having the amino acid sequence of SEQ ID NO:9.

Similarly, a “crystal-forming fragment” of a Cry protein is a fragment of any of the known Cry proteins (i.e., less than full length of the wild-type Cry protein) that still retains the ability of self-crystallization, which is demonstrated both by crystallization by the fragment alone and by causing a fusion protein to self-crystallize when the fragment is present in the fusion protein with another protein of interest (e.g., an enzyme). In addition to being a truncated form of a Cry protein, a “crystal-forming fragment” may further contain one or more modifications to the native amino acid sequence such as insertions, deletions, or substitutions, especially conservative modifications, such that the resultant “crystal-forming fragment” shares an amino acid sequence identity of at least 80%, 81%, 82%, 83%, 84%, 88%, 89%, 90%, 91%, 92%, 93%, 94%, 95%, 96%, 97%, 98%, 99%, or 99.5% to the polypeptide sequence of the corresponding fragment of a wild-type Cry protein. Exemplary crystal-forming fragments of a Cry protein have been described in earlier disclosures, e.g., WO2018/028371.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

There are various known methods in the art that permit the incorporation of an unnatural amino acid derivative or analog into a polypeptide chain in a site-specific manner, see, e.g., WO 02/086075.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

As used in herein, the terms “identical” or percent “identity,” in the context of describing two or more polynucleotide or amino acid sequences, refer to two or more sequences or subsequences that are the same or have a specified percentage of amino acid residues or nucleotides that are the same (for example, a Cry protein or a crystal-forming fragment of a Cry protein sequence comprised in the fusion protein of this invention has at least 80% identity, preferably 85%, 90%, 91%, 92%, 93, 94%, 95%, 96%, 97%, 98%, 99%, or 100% identity, to a reference sequence, e.g., the amino acid sequence of a corresponding wild-type Cry protein or fragment), when compared and aligned for maximum correspondence over a comparison window, or designated region as measured using one of the following sequence comparison algorithms or by manual alignment and visual inspection. Such sequences are then said to be “substantially identical.” With regard to polynucleotide sequences, this definition also refers to the complement of a test sequence. Preferably, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection, see, e.g., Current Protocols in Molecular Biology (Ausubel et al., eds. 1995 supplement).

Examples of algorithms that are suitable for determining percent sequence identity and sequence similarity are the BLAST and BLAST 2.0 algorithms, which are described in Altschul et al., (1990) J. Mol. Biol. 215: 403-410 and Altschul et al. (1977) Nucleic Acids Res. 25: 3389-3402, respectively. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The term “recombinant” when used with reference, e.g., to a cell, or a nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral vector derived from a viral genome, or nucleic acid fragment/construct. Typically, an expression cassette includes a polynucleotide to be transcribed, operably linked to a promoter. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

A “promoter” is defined as an array of nucleic acid control sequences that direct transcription of a polynucleotide sequence. As used herein, a promoter includes necessary polynucleotide sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a polynucleotide expression control sequence (such as a promoter, or array of transcription factor binding sites) and a second polynucleotide sequence, wherein the expression control sequence directs transcription of the polynucleotide sequence corresponding to the second sequence.

The term “heterologous” as used in the context of describing the relative location of two elements, refers to the two elements such as polynucleotide sequences (e.g., a promoter or a protein/polypeptide-encoding sequence) or polypeptide sequences (e.g., a Cry protein sequence or another polypeptide sequence) that are not naturally found in the same relative positions. Thus, a “heterologous promoter” of a gene refers to a promoter that is not naturally operably linked to that gene. Similarly, a “heterologous polypeptide” or “heterologous polynucleotide” to a Cry protein or its encoding sequence or a fragment thereof is one derived from an origin other than the Cry protein or, in the case of a fragment of a Cry protein/coding sequence, may be derived from another part of the same Cry protein or coding sequence, but not naturally connected to the fragment in the same fashion. The fusion of a fragment of a Cry protein (or its coding sequence) with a heterologous polypeptide (or polynucleotide sequence) does not result in a longer polypeptide or polynucleotide sequence that can be found naturally in the wild-type Cry protein.

By “host cell” is meant a cell that contains an expression vector and supports the replication or expression of one or more coding sequences harbored in the expression vector. Host cells may be prokaryotic cells such as Bacillus thuringiensis (Bt), Bacillus subtilis (Bs), or E. coli, or eukaryotic cells such as yeast, insect, amphibian, or mammalian cells such as CHO, HeLa and the like, e.g., cultured cells, explants, and cells in vivo.

The term “about” as used herein denotes a range of +/−10% of a reference value. For examples, “about 10” defines a range of 9 to 11.

### DETAILED DESCRIPTION OF THE INVENTION

### I. Introduction

There has been growing interest in using enzymes to catalyze industrial reactions due to their high reactivity, excellent regio- and enantiospecificity, and low environmental toxicity. In order to financially compete with chemical catalysis, biocatalysts are optimized so they can be recycled multiple times. Additionally, biocatalysts are generally optimized so they can withstand high concentrations of organic solvents—conditions that can promote substrate solubility and enzyme activity. Enzyme immobilization can facilitate easy removal and subsequent reuse of enzymes during multiple rounds of catalysis, in addition to improving enzyme stability resistant to harsh industrial conditions. Immobilization by entrapment is particularly attractive since it does not involve any modifications to the enzyme structure, increasing the chance for high activity retention and native enzyme conformation. In this disclosure, a novel one-step method is described for producing entrapped recombinant proteins (especially enzymes, including multi-enzymes) in protein crystals.

To date, all entrapment methods involve first producing the enzyme and carrier separately, and then mixing them to generate the immobilized enzyme. This multi-step process requires purifying and concentrating the enzyme, synthesizing the carrier, and then mixing them, usually including the use of a catalyst to initiate the polymerization of the carrier. This procedure is tedious and expensive. A method that generates an entrapped enzyme or multi-enzyme system in one-step process can significantly reduce production costs and lead to cheaper commercial catalysts. The method of this invention leads to the entrapped enzyme in one step, avoiding the need for purification of the free enzyme or polymerization of the carrier. Minimizing time, processes, and materials for producing enzyme catalysts allows for greener and more cost-effective products.

### II. Production of Recombinant Proteins

### A. General Recombinant Technology

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, J. Chrom. 255: 137-149 (1983).

The sequence of a gene of interest, such as the polynucleotide sequence encoding an enzyme like lipase or hydrolase, a polynucleotide encoding a Cry protein or a crystal-forming fragment thereof, and synthetic oligonucleotides can be verified after cloning or subcloning using, e.g., the chain termination method for sequencing double-stranded templates of Wallace et al., Gene 16: 21-26 (1981).

### B. Coding Sequences

Polynucleotide sequences encoding Cry proteins, fragments, or fusion proteins for use in this invention can be readily constructed by using the corresponding coding sequences for the Cry proteins, fragments, or combining the coding sequences for the fusion partners, such as a Cry3Aa protein and Bacillus subtilis lipase A (lipA). The sequences for Cry proteins and enzymes are generally known and may be obtained from a commercial supplier.

In addition to the use of full length wild-type Cry proteins for producing crystal-forming proteins for use in this invention, fragments of Cry proteins and/or variants of Cry proteins may also be useful. A DNA sequence encoding a Cry protein can be modified to generate fragments or variants of the Cry protein. So long as the fragments and variants retain the ability to spontaneously form crystals when expressed in a host cell, especially a Bacillus bacterial cell, they can be used for producing the protein crystals, either by themselves or by way of fusion proteins capable of undergoing spontaneous crystallization, and therefore producing protein crystals containing one or more recombinant proteins (e.g., one or more enzymes) embedded within. Typically, the variants bear a high percentage of sequence identity (e.g., at least 80, 85, 90, 95, 97, 98, 99% or higher) to the wild-type Cry protein sequence, whereas the fragments may be substantially shorter than the full length Cry protein, such as having some amino acids (e.g., 10-300 or 20-200 or 50-100 amino acids) removed from the N- or C-terminus of the full length Cry protein. For example, a useful Cry3Aa fragment may be as short as the first 290 amino acids from the N-terminus, encompassing Domain I of the protein. Other examples of such fragments include a Cry protein fragment having its first 57 amino acids from N-terminus removed and a Cry protein fragment having its C-terminal 18 amino acids removed. The ability of a recombinantly produced Cry protein, a fragment thereof, or a fusion protein comprising a Cry protein or fragment to undergo spontaneous crystallization can be verified by electron micrograph, whereas the enzymatic activity of a recombinantly produced enzyme, including in the form of a fusion protein with a Cry protein or a fragment thereof, can be confirmed by established assays for each specific enzyme. Exemplary Cry protein fragments capable of self-crystalizing can be found in the inventors' earlier publications, e.g., WO2018/028371.

In the case of a fusion protein, a peptide linker or spacer is used between the coding sequences for a Cry protein/fragment and one or more heterologous polypeptides. One purpose is to ensure the proper reading frame for the fusion protein such that the coding sequences for both Cry protein/fragment and the heterologous polypeptide(s) are in frame. Another purpose is to provide appropriate spatial relationship between the Cry protein/fragment and the heterologous polypeptide(s), such that each component of the fusion protein may retain its original functionality: the Cry protein/fragment is able to cause self-crystallization of the fusion protein, and the heterologous polypeptide such as an enzyme remains active in its catalytic capacity. Also, one or more linkers may be placed at the very beginning and/or the very end of the open reading frame, so as to facilitate proper start and termination of the coding sequence translation. Such linkage amino acid sequences are usually shorts and typically no longer than 100 or 50 amino acids, such as between 1 to 100, 1 or 2 to 50, 2 or 3 to 25, 3 or 4 to 10 amino acids.

### C. Sequence Modification for Preferred Codon Usage in a Host Organism

The polynucleotide sequence encoding a recombinant protein to be expressed according to the method of this invention can be further altered to coincide with the preferred codon usage of a particular host. For example, the preferred codon usage of one strain of bacterial cells can be used to derive a polynucleotide that encodes a recombinant polypeptide of the invention and includes the codons favored by this strain. The frequency of preferred codon usage exhibited by a host cell can be calculated by averaging frequency of preferred codon usage in a large number of genes expressed by the host cell (e.g., calculation service is available from web site of the Kazusa DNA Research Institute, Japan). This analysis is preferably limited to genes that are highly expressed by the host cell.

At the completion of modification, the coding sequences are verified by sequencing and are then subcloned into an appropriate expression vector for recombinant production of a protein (e.g., an enzyme such as a lipase) along with a Cry protein, a crystal-forming fragment of the Cry protein, or a fusion protein comprising the Cry protein or crystal-forming fragment, such that the protein (e.g., enzyme) is produced and embedded in the protein crystals formed by the Cry protein, fragment, or fusion protein.

### III. Expression and Isolation of Proteins Embedded in Protein Crystals

Following verification of the coding sequence, co-expression of a protein of interest (such as an enzyme) and a Cry protein, a crystal-forming fragment thereof, or a fusion protein comprising the Cry protein or fragment of this invention can be produced using routine techniques in the field of recombinant genetics, relying on the polynucleotide sequences encoding the Cry fusion protein disclosed herein.

### A. Expression Systems

To obtain high level expression of a polynucleotide sequence encoding a recombinant protein, one typically subclones a polynucleotide encoding the protein in the correct reading frame into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra. Bacterial expression systems for expressing the polypeptide are available in, e.g., E. coli, Bacillus sp., Salmonella, and Caulobacter. Kits for such expression systems are commercially available.

The promoter used to direct expression of a recombinant protein depends on the particular host cells used for the recombinant protein production. For instance, for effective expression in a Bacillus bacterial strain such as Bacillus thuringiensis (Bt) or Bacillus subtilis (Bs) cells, a promoter known to direct robust protein expression in these particular bacterial cells should be chosen. As shown in the Examples of this disclosure, two separate promoters, cyt1Aa and cry3Aa, have been successfully used to direct the expression of a recombinant protein as well as a Cry protein. The promoter is optionally positioned about the same distance from an exogenous or recombinant protein transcription start site as it is from the transcription start site in its natural setting. As is known in the art, however, some variation in this distance can be accommodated without loss of promoter function. In some cases, a constitutive promoter is used, whereas in other cases an inducible promoter rather than a constitutive promoter is preferred. Further, the placement of a common promoter or multiple separate promoters (which may be the same or different kind of promoters) and/or the placement of one or more separate ribosome binding sites separating different segments of coding sequences (each encoding a recombinant protein or a crystal-forming protein) in a common expression cassette (e.g., an expression vector such as a plasmid) can allow different expression ratios of the recombinant protein(s) to the crystal-forming protein so as to maximize the percentage of the recombinant protein(s) being entrapped or immobilized within the protein crystals formed by the crystal-forming protein such as a Cry protein, a crystal-forming fragment thereof, or a fusion protein comprising the Cry protein or the fragment.

In addition to the promoter, the expression vector typically includes a transcription unit or expression cassette containing all the additional elements that are required for the expression of the recombinant protein(s) and the crystal-forming protein in host cells. A typical expression cassette thus contains a promoter operably linked to the polynucleotide sequence encoding the recombinant protein and/or crystal-forming protein and signals required for efficient polyadenylation of the transcript, ribosome binding sites, and translation termination. The polynucleotide coding sequence may be linked to a cleavable signal peptide sequence to promote secretion of the polypeptide by the transformed cell. Such signal peptides include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

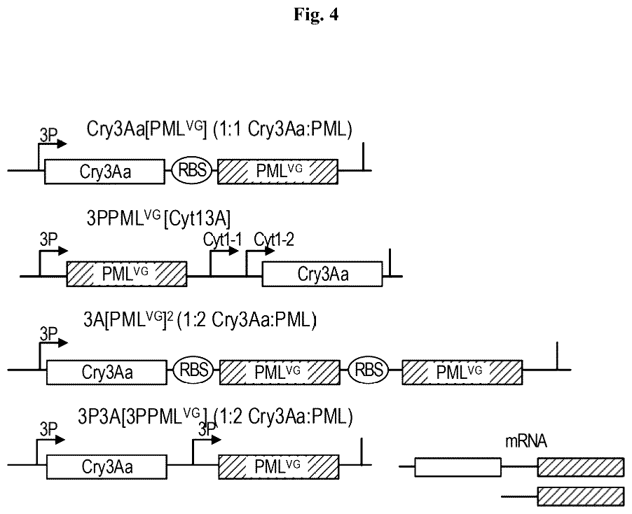

In addition to a promoter sequence, the expression cassette should also contain a transcription termination region downstream of the coding sequence to provide for efficient termination. The termination region may be obtained from the same gene as the promoter sequence or may be obtained from different genes. The placement of a commonly shared termination site in combination with the placement of multiple promoters directing transcription of multiple coding sequences can also be used to adjust the expression ratios of a recombinant protein to a crystal-forming protein, see, e.g., illustration in FIG. 4.

The particular expression vector used to transport the genetic information into the cell is not particularly critical. Any of the conventional vectors used for expression in eukaryotic or prokaryotic cells may be used, especially those suitable for expression in cells of Bacillus sp. such as Bt and Bs. Standard bacterial expression vectors include plasmids such as pBR322 based plasmids, pSKF, pET23D, and fusion expression systems such as GST and LacZ.

The elements that are typically included in expression vectors also include a replicon that functions in bacteria such as Bacillus sp. and E. coli, a gene encoding antibiotic resistance to permit selection of bacteria that harbor recombinant plasmids, and unique restriction sites in nonessential regions of the plasmid to allow insertion of coding sequences. The particular antibiotic resistance gene chosen is not critical, any of the many resistance genes known in the art are suitable. Similar to antibiotic resistance selection markers, metabolic selection markers based on known metabolic pathways may also be used as a means for selecting transformed host cells.

### B. Transfection Methods

Standard transfection methods are used to produce bacterial, mammalian, yeast, insect, or plant cell lines that express large quantities of a recombinant fusion protein of this invention, which are then purified using standard techniques (see, e.g., Colley et al., J. Biol. Chem. 264: 17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132: 349-351 (1977); Clark-Curtiss & Curtiss, Methods in Enzymology 101: 347-362 (Wu et al., eds, 1983).

Any of the well-known procedures for introducing foreign polynucleotide sequences into host cells may be used. These include the use of calcium phosphate transfection, polybrene, protoplast fusion, electroporation, liposomes, microinjection, plasma vectors, viral vectors and any of the other well-known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the recombinant protein(s) along with a crystal-forming protein (e.g., a Cry protein, a crystal-forming fragment thereof, or a fusion protein comprising a Cry protein or a crystal-forming fragment thereof) according to the method of this invention. As described above and herein, the recombinant protein or proteins (e.g., enzyme or enzymes) and the crystal-forming protein may be contained within one single expression cassette, e.g., in the same expression vector, where all coding sequences may be under the control of one single promoter, or each coding sequence may be under the control of a separate promoter (which optionally may differ from one another). In the alternative, each one of the coding sequences may be present in a separate expression cassette (e.g., expression vector). In either alternative, different ratios of the recombinant protein(s) to the crystal-forming protein may be achieved by using single copy or multiple copies of any one coding sequence or by placing separate or commonly shared ribosome binding site(s) and/or termination site(s) in the expression cassette, see, e.g., illustration in FIG. 4.

### C. Isolation of Crystal-Entrapped Recombinant Proteins

Once the expression of the recombinant protein(s) along with a crystal-forming protein (e.g., a Cry protein, a crystal-forming fragment thereof, or a fusion protein comprising a Cry protein or a crystal-forming fragment thereof) in transfected host cells is confirmed, e.g., via electron micrograph for detecting protein crystals or an immunoassay such as Western blotting analysis, the host cells are then cultured in an appropriate scale for the purpose of purifying or isolating the recombinant protein entrapped within the protein crystals formed by the Cry protein, a crystal-forming fragment thereof, or a fusion protein comprising a Cry protein or a crystal-forming fragment thereof.

When the recombinant protein(s) and the crystal-forming protein are produced recombinantly by transformed bacteria in large amounts, for example after promoter induction, the recombinant protein(s) become entrapped within the crystals formed by the crystal-forming protein. In other words, the recombinantly produced proteins are present in crystalline form or insoluble aggregates within the host cells. Thus, one can readily isolate the crystals from the cell lysate based on their distinct density by utilizing techniques such as centrifugation and density gradient separation followed by one or more rinsing steps to further remove contaminants from the protein crystals.

There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells, e.g., by incubation in a buffer of about 100-150 μg/ml lysozyme and 0.10% Nonidet P40, a non-ionic detergent. The cell suspension can be ground using a Polytron grinder (Brinkman Instruments, Westbury, NY). Alternatively, the cells can be sonicated on ice. Additional methods of lysing bacteria are described in Ausubel et al. and Sambrook and Russell, both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Upon isolation, the recombinant protein(s) recovered from host cells in the form of protein crystals, the protein or proteins may be directly used according to their inherent biological activity: for example, a lipase entrapped in Cry protein crystals may be used in a reaction to hydrolyze triglycerides. By virtue of being in an insoluble crystal form, the lipase has a heighted level of resistance to harsh environmental conditions such as high temperature, extreme pHs, organic solvents, etc., thus allowing repeated cycles of cleaning and reuse.

In the alternative, following the washing step, the inclusion bodies are solubilized to release the entrapped recombinant protein(s) by the addition of a solvent that is both a strong hydrogen acceptor and a strong hydrogen donor (or a combination of solvents each having one of these properties). The protein(s) from the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, may be inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein(s) can be separated from other bacterial proteins by standard separation techniques. For further description of purifying recombinant polypeptides from bacterial inclusion body, see, e.g., Patra et al., Protein Expression and Purification 18: 182-190 (2000).

While the protein crystals tend to remain insoluble at lower or neutral pHs, placing them in alkaline solutions with pH at or greater than 10 or 11 can often effectively dissolve the protein. Once dissolved, the protein can then be analyzed by gel separation (e.g., on an SDS gel) and immunoassays to confirm its identity based on the appropriate molecular weight and immunoreactivity.

### IV. Applications of Immobilized Proteins

Another aspect of the present invention relates to the use of a recombinant protein, especially an enzyme, entrapped and immobilized in protein crystals produced according to the methods described herein to exert the protein's inherent biological activity, for example, to perform reactions typically catalyzed by the enzyme present in the protein crystals, such as hydrolysis, esterification, ligation, proteolysis, and the like. As organic solvents are often able to facilitate such reactions and the immobilized recombinant protein produced by the method of this invention is highly tolerant to the presence of organic solvents, a reaction performed using the immobilized protein according to this invention often not only a water-based solvent but also one or more organic solvents, e.g., ethanol, methanol, acetonitrile, and dimethylformamide.

As the inventors discovered that immobilization of recombinant protein(s) within the crystalline Cry protein or fusion proteins leads the protein(s) to have a higher level of resistance to organic solvents and a higher level of thermostability, potentially can retain enzymatic activity for use in more cycles of reactions. In some cases, this reaction process includes a cleaning step, performed after the completion of one round of the reaction and removal of the reaction product(s) as well as any remaining substrate, during which the protein crystals containing recombinant protein(s) are rinsed or washed in preparation of being used again with fresh substrate in a subsequent round of reaction.

### EXAMPLES

The following examples are provided by way of illustration only and not by way of limitation. Those of skill in the art will readily recognize a variety of non-critical parameters that could be changed or modified to yield essentially the same or similar results.

### Example 1

### Introduction

### Immobilization of Enzymes

The major bottleneck to the practical use of enzymes as industrial catalysts is the cost of production and useable lifetime. While biomolecular engineering methods can be used to enhance enzyme stability, these techniques do not aid in rapid production and isolation, particularly when high purities are necessary. A cost-effective biocatalyst should be able to be recycled over many reaction cycles, with minimal loss of activity. One method to improve enzyme stability and make them reusable is immobilization, either by covalent attachment to beads, or by adsorption into porous materials. Immobilization of enzymes allows them to be easily filtered and reused over successive reaction cycles, and in some cases can lead to increased temperature stability and/or organic solvent tolerance due to the reduced conformational flexibility of the enzyme in the matrix¹. This rigidity is also beneficial to mechanical stability by helping to protect against the constant agitation that occurs in large-scale reactors.²

A major limitation of both bead attachment and porous material adsorption approaches, however, is that ˜90-99% of the biocatalyst composite is inactive, greatly reducing the catalytic productivity per weight.³ Furthermore, these approaches require multiple steps: (1) production and purification of the enzyme catalyst, (2) production of the support, and (3) anchoring of the enzyme catalyst on the support. These steps can add significantly to the cost and impact catalytic activity. If a strategy can be developed to generate and isolate an immobilized lipase in a single step, it can dramatically lower the cost of the catalyst.

### Genetically-Encoded Immobilized Lipases

Genetically-encoded, or in-vivo, immobilization approaches that involve the direct production of immobilized enzymes in bacterial cells, represents a promising direction for more efficient and economical biocatalyst production. Consolidating expression and immobilization into a single step removes the need of columns for enzyme purification, as well as reagents to introduce reactive chemistries, greatly reducing time and production costs.

One of the earliest reports of producing active enzyme particles in cells came from the work of Worrall et al., who showed that they could produce catalytically active inclusion bodies (CatIBs) of β-galactosidase in E. coli.⁴ This discovery upended the paradigm that inclusion bodies (IBs) were inactive waste species in cells and prompted the idea that IBs could be used as immobilized catalysts. Since most enzymes are naturally soluble, producing CatIBs can be a challenge. Thus most approaches to produce in vivo immobilized enzymes have involved the incorporation of a fusion-tag to promote aggregation.

Several fusion tags have been exploited to generate immobilized enzymes in vivo including self-aggregating peptides and protein domains. Peptide tags such as ELK16 and L₆KD have been successfully fused to target enzymes to drive in vivo aggregation. While the use of tags resulted in the formation of highly active enzyme aggregates, none of the these aggregates was shown to be reusable.^{5,6} Similarly, several protein domains have been used to promote in vivo enzyme immobilization in cells.⁷⁻⁹ The most successful example has been the work of Diener et al. who used the short coiled-coil domain TDoT of the cell-surface protein tetrabrachion from the hyperthermophilic archaeon Staphylothermus marinus to immobilize several enzymes.^{10,11} While enzymes immobilized by the TDoT tag displayed good recyclability, the aggregates produced generally had low activity due to diffusion limitations. Identifying a platform capable of promoting the production of in vivo immobilized enzymes with both high activity and good reusability had until recently, remained elusive.

### Cry-Enzyme Fusion Proteins as Genetically-Encoded Immobilized Catalysts

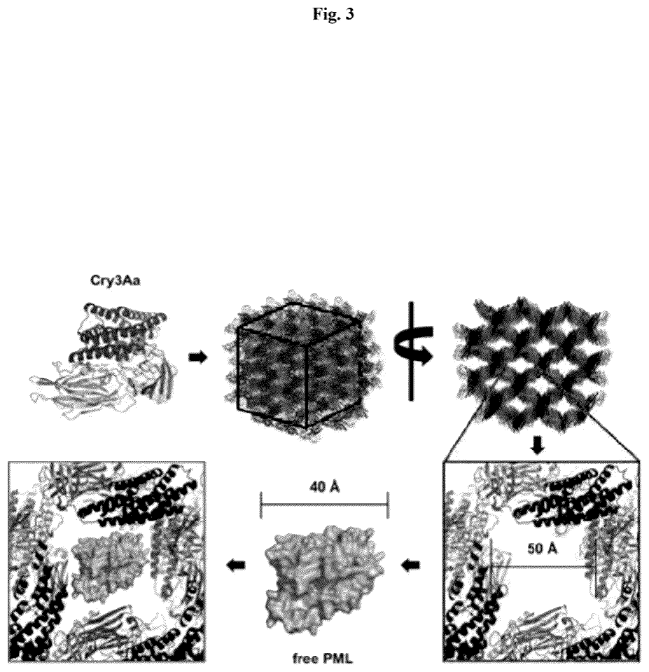

Recently a new strategy was developed to produce genetically-encoded immobilized enzymes based on crystal proteins (Cry), such as Cry3Aa. Cry proteins are insecticidal proteins that innately form crystals inside the bacterium Bacillus thuringiensis (Bt) (FIG. 3).¹²⁻¹⁴ It was previously demonstrated that expression of Cry3Aa fused to GFP or mCherry yielded fluorescent protein crystals in Bt—indicating that the fusion partners were folded and functional.¹⁵ Electron microscopy carried out on Cry3Aa crystals and Cry3Aa-GFP crystals revealed them to be similar in size, indicating that the fusion partner does not change the crystal packing (FIG. 3b,c).¹⁵

The use of this Cry crystal platform to produce genetically-immobilized lipases as potential biodiesel catalysts was explored, see, e.g., WO2018/028371. The first-generation biodiesel catalyst was generated by genetically fusing Bacillus subtilis lipase A (lipA) to the C-terminus of a truncated Cry3Aa (Cry3Aa*) and then producing the fusion protein in Bt. The resultant Cry3Aa*-lipA fusion crystals exhibited higher catalytic activities compared to free lipA, as well as improved solvent and temperature stability.¹⁶ Significantly, the Cry3Aa*-lipA fusion crystals can be used to produce biodiesel from coconut oil with high conversion efficiency over 10 reaction cycles (FIG. 4). These properties made the Cry3Aa*-lipA catalyst the first example of a genetically-encoded immobilized enzyme that retained both high activity and recyclability.

### Cry3Aa as a Carrier for Enzyme Entrapment

A potential limitation of using Cry3Aa-fusion crystals, however, is that the enzyme of interest is chemically modified at its N-terminus, which can hamper enzyme activity. Moreover, fusion to a bulky Cry3Aa protein may cause problems to the enzyme during folding. Indeed, Cry3Aa-fusion enzymes of a Proteus mirabilis lipase have been generated, which only retained 5% activity of retention compared to wild-type. This motivated further efforts to explore novel approaches to produced immobilized enzymes using Cry3Aa.

Cry3Aa proteins crystals contain large porous 50 Å×50 Å channels. It is envisaged that these channels can act as cages to trap the enzyme of interest inside (FIG. 3). Since Cry3Aa forms a crystal the target enzyme should be spatially confined and ordered such that each monomer does not interfere with the other. Moreover, since the unit cell dimensions of Cry3Aa are known, one can quantify the amount of enzyme molecules loaded into the Cry3Aa crystals, a characteristic can be measured using the Cry3Aa-fusion crystals based on the structural information. Most importantly, this strategy retains the feature of a one-step immobilization platform, and the entrapped enzyme is much more likely to be in its native state since there are no covalent modifications.

### Results and Discussions

### Entrapment of PML Inside Cry3Aa Crystals

Proteus mirabilis lipase (PML) is a bacterial lipase capable conversion of vegetable oils including waste cooking oil to biodiesel. PML is highly soluble and expresses well in E. coli. The potential of PML fused to Cry3Aa was recently explored as a catalyst for biodiesel production from canola oil and waste cooking oil, and an optimized mutant containing two-point mutations (I118V, E130G, PML^{VG}) was produced. PML is a good target for Cry3Aa entrapment since the fusion of PML to Cry3Aa drastically reduces the activity. Additionally, the size of PML (55 Å×41 Å×26 Å) is a good fit for the Cry3Aa channel (FIG. 1). For clarity, square brackets ([ and]) are used to indicate the entrapment of the PML molecules (e.g., Cry3Aa[PML]), whereas a hyphen (-) indicates a genetic fusion (e.g., Cry3Aa-PML).

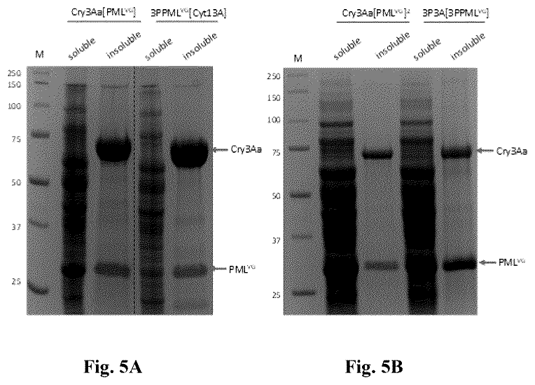

Several plasmid variations were generated to improve the amount of PML loaded into the crystals. A list of these constructs is displayed in FIG. 4. The initial Cry3Aa[PML] coexpression cassette was generated by cloning the PML gene containing a separate ribosome binding site downstream of Cry3Aa. This plasmid was transformed, expressed and the resulting crystals were purified as described previously.¹⁵ Solubilization of the crystals and analysis by SDS-PAGE indicated that the Cry3Aa crystals contained PML (FIG. 5a).

Several methods were tried to improve the PML loading into the Cry3Aa crystals. First, Cry3Aa was put under the control of two cyt1Aa promoters¹⁷ and PML under one cry3Aa promoter (3PPML^{VG}[Cyt13A]). It was anticipated that the temporal difference between the promoters (cyt1Aa is activated during late-stage sporulation while cry3Aa is activated during vegetation and early-stage sporulation) would cause PML to accumulate in the cell first and increase the relative ratio of PML to Cry3Aa during crystallization. Although PML entrapment was achieved, the loading was much lower than Cry3Aa[PML^{VG}] (FIG. 5a).

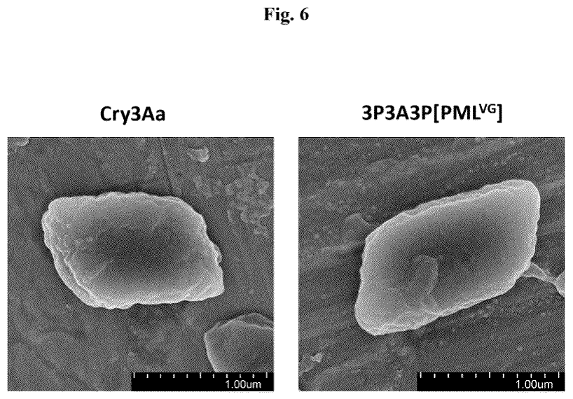

Another approach was based on increasing the ratio of PML to Cry3Aa in the transcript. This was accomplished implementing two strategies. First, by inserting another copy of PML downstream of Cry3Aa[PML^{VG}] making Cry3Aa[PML^{VG}]². Since the proteins are under one promoter, each transcript will have double the amount of PML per Cry3Aa. Second, by putting Cry3Aa and PML under two separate cry3Aa promoters but only one terminator (3P3A[3PPML^{VG}]). In the latter case, for each transcript containing Cry3Aa there will be two transcripts containing PML. Each of these strategies should result in a theoretical 2:1 PML:Cry3Aa ratio. Both constructs resulted in higher loading of PML into the Cry3Aa crystals, albeit 3P3A[3PPML^{VG}] demonstrated the highest loading efficiency at 64-75% depending on the batch (FIG. 5b). This corresponds to around 4×10⁵ to 6×10⁵ molecules of PML^{VG} per crystal, which is unprecedented in terms of enzyme loading. Notably, these crystals retained near identical morphology and size to native Cry3Aa crystals (FIG. 6).

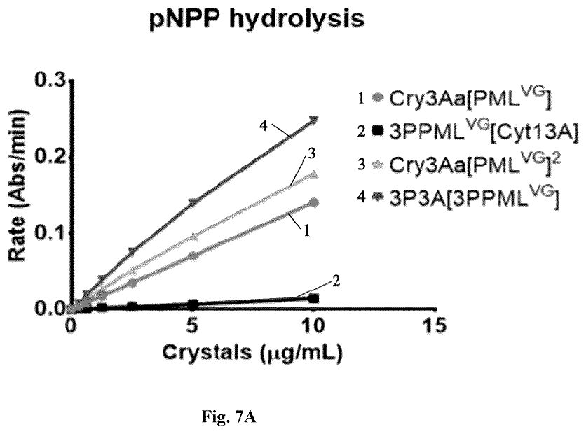

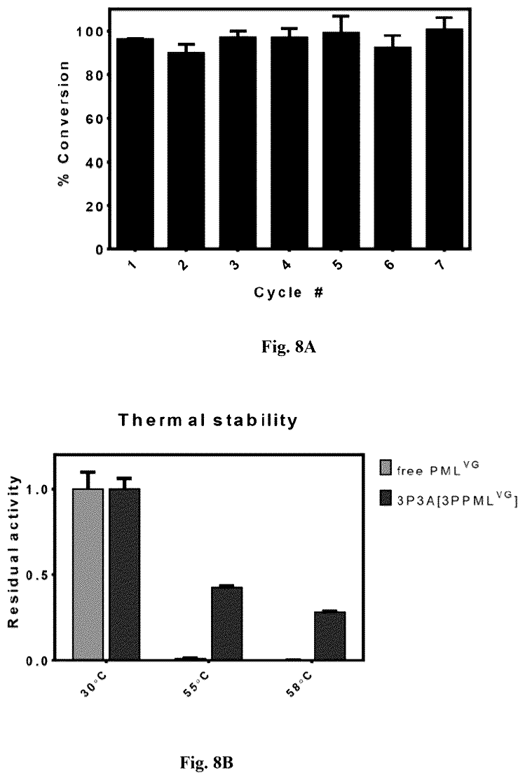

The activities of all the resulting PML entrapped Cry3Aa crystals were determined by measuring the hydrolysis of p-nitrophenyl palmitate. As demonstrated in FIG. 7, higher PML loading into the crystals resulted in higher activity per μg crystal, with the 3P3A3P[PML^{VG}] construct displaying the highest activity. Moreover, entrapment of PML^{VG} inside Cry3Aa crystals significantly stabilized it against thermal denaturation, displaying a 200-fold increase in residual activity after incubation for 1 h incubation at 58° C. (FIG. 8B).

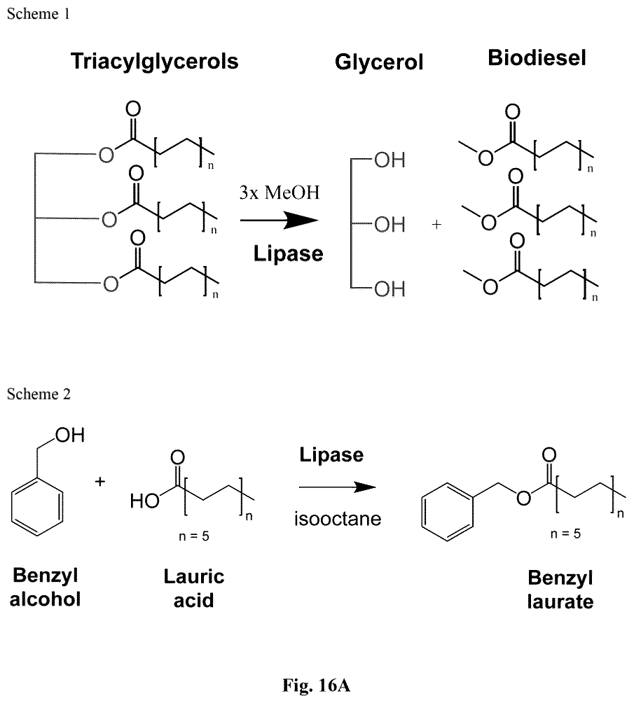

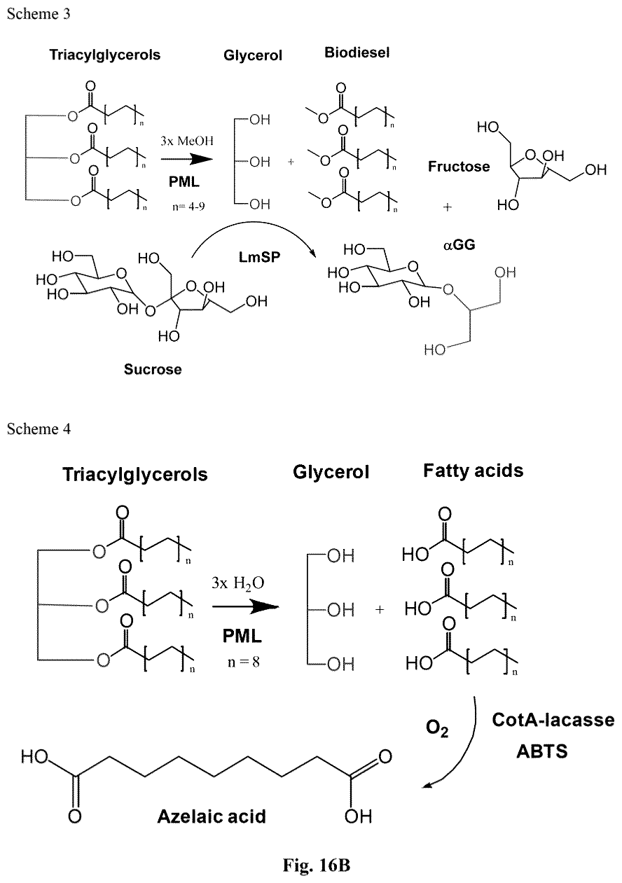

With the optimized immobilized lipase at hand, the practicality of the catalyst for industrial reactions was tested. Two common applications of lipases are biodiesel production (Scheme 1 in FIG. 16A) and ester synthesis in organic solvents such as the cosmetic compound benzyl laurate (Scheme 2 in FIG. 16A).

The optimized Cry3Aa[PML^{VG}] crystals were capable of converting waste cooking oil into biodiesel in 10 h and retained maximal conversion for 7 reaction cycles (FIG. 8A). These data support the high catalytic activity and stability of the immobilized catalyst for biodiesel production.

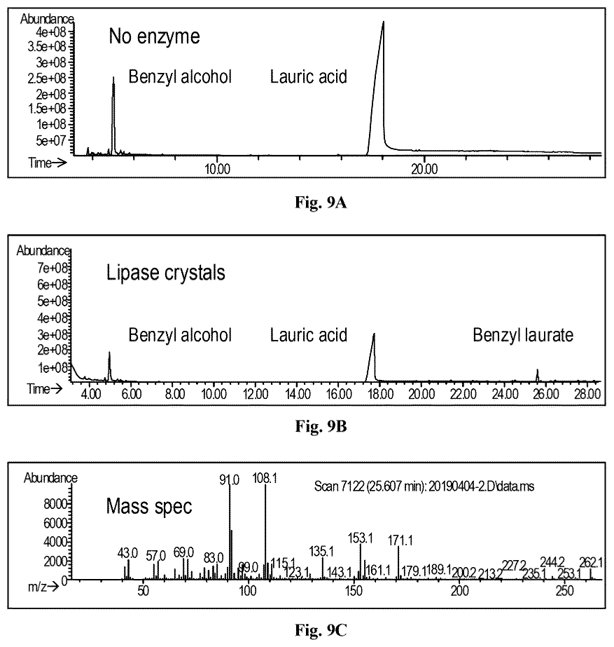

For benzyl laurate synthesis, the optimized Cry3Aa[PML^{VG}] crystals were lyophilized in water for 24 h. The dried crystals were then reacted with 10 mM benzyl alcohol and 10 mM lauric acid in neat isooctane for 24 h. Gas chromatography-mass spectrometry (GCMS) analysis of the reaction mixture revealed that benzyl laurate was produced (FIG. 9). While this reaction has not been optimized to maximize yield, it demonstrates that the Cry3Aa entrapped crystals can be used in neat organic solvents for synthesis reactions.

As further demonstration that PML can be entrapped within Cry3Aa crystals, in vitro co-crystallization of carboxy-rhodamine-labelled PML and Cry3Aa was performed by vapor diffusion (FIG. 10). Since only Cry3Aa exhibits fluorescence upon UV (280 nm) irradiation, and PML is dye-labelled, formation of Cry3Aa-entrapped PML crystals could be confirmed based on their fluorescent properties (FIG. 10). Notably, these data demonstrate that Cry3Aa-mediated entrapment can also be performed in vitro.

### Coentrapment of Multiple Proteins Inside Cry3Aa Crystals

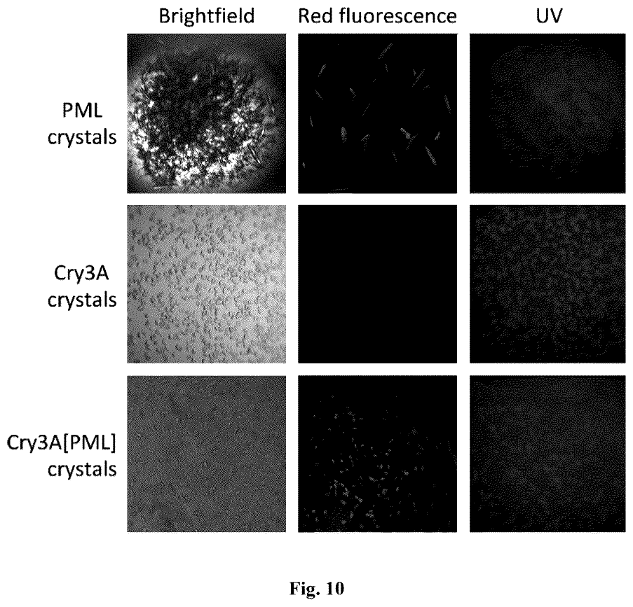

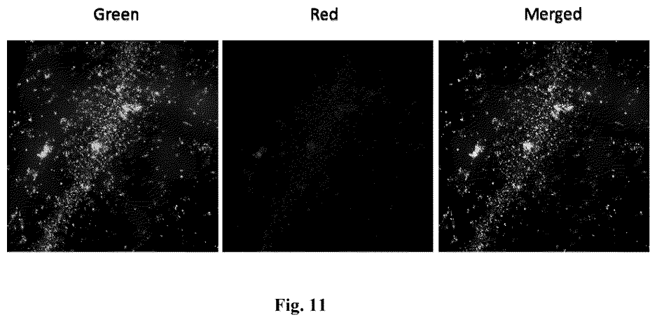

One major advantage of using enzyme over chemical catalysis is that enzymes are specific, allowing for multiple reactions to occur in one-pot without unwanted side reactions or side products. It was hypothesized using the expression platform one could entrap multiple enzymes simultaneously in a single crystal. Notably, this would offer a significant advantage over other multi-enzyme systems since there is no need to purify the enzymes separately. As a proof of concept, mCherry, green fluorescent protein (GFP) and Cry3Aa were incorporated on the same plasmid and expressed in Bt. Cry3Aa was expressed under one Cry3Aa promoter, while GFP and mCherry were expressed on a separate Cry3Aa promoter separated by a ribosome binding site. Crystals produced contained both red and green fluorescence indicating co-entrapment of both proteins occurred in a single crystal (FIG. 11).

Co-immobilizing two or more enzymes on a single platform can be beneficial to either impart the catalyst with a broader substrate specificity or generate a coupled system that promotes the synthesis of higher value products. In the latter case, active site proximity can lead to much faster turnover rates since intermediates are released in close proximity to the subsequent active site, so that they can be quickly turned over before diffusing away.

Glycerol—a byproduct of FAME biodiesel production is usually extracted from the aqueous layer and purified prior to its separate commercialization. A more efficient and value-added approach would be to simultaneously convert glycerol to a cosmetic product during the transesterification process. Not only would this save time by doing both reactions in one pot, but also removal of glycerol might increase the efficiency and yield of FAME biodiesel by pulling the equilibrium of transesterification reaction towards the products via Le Chatelier's principle.

Among glycerol-derived cosmetics, glycosylated glycerol such as 2-O-(α-D-gluco-pyranosyl)-sn-glycerol (αGG) has gained a lot of attention as a powerful moisturizing agent.³⁰ Interestingly, Goedl et al. demonstrated that they could produce this compound enzymatically from sucrose and glycerol using a sucrose phosphorylase enzyme from Leuconostoc mesenteroides (LmSP).³⁰ Therefore, it would be plausible to simultaneously convert oil to biodiesel and αGG using a one-pot combination of PML and LmSP (Scheme 3 in FIG. 16B).

Having already entrapped PML inside Cry3Aa crystals, the inventors' next step was to first verify that LmSP can be entrapped inside Cry3Aa crystals as well. A plasmid was constructed that coexpresses Cry3Aa and LmSP on separate cry3Aa promoters. This plasmid was transformed and expressed in Bt. SDS-PAGE analysis of the crystals indicated that LmSP was entrapped inside Cry3Aa crystals with high efficiency—33% of the protein molecules in the crystals are LmSP (FIG. 12a). For the LmSP:PML dual catalyst construct, a copy of PML^{VG} was inserted downstream of LmSP with its own ribosome binding site. After expression of this new construct 3P3A3P[LmSP][PML^{VG}] in Bt and purification, SDS-PAGE analysis revealed that both LmSP and PML^{VG} were entrapped in Cry3Aa crystals with high efficiency (FIG. 12a). These 3P3A3P[LmSP][PML^{VG}] crystals displayed both high sucrose phosphorylase activity (FIG. 12b) and pNPP hydrolysis activity (FIG. 12c).

### Entrapment of PML Inside Cry3Aa* Crystals

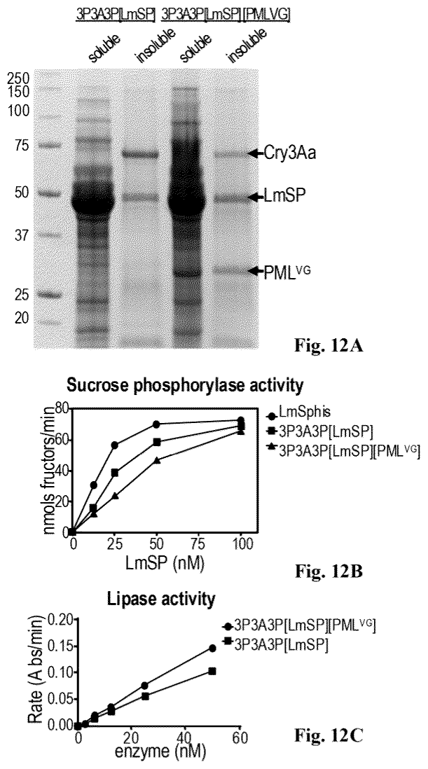

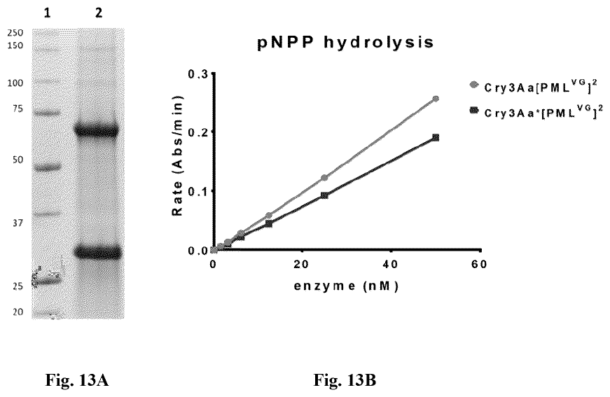

A potential limitation of using native Cry3Aa crystals is that they are soluble at alkaline pH. Thus, reactions performed at alkaline pH will cause Cry3Aa to solubilize and release the entrapped protein or enzyme. It was previously demonstrated that truncation of 18 amino acids from the C-terminus of Cry3Aa and expression in Bt resulted in the production of particles that were much more resistant to solubilization. It was hypothesized that if these C-terminally truncated crystals (Cry3Aa*) still retained pores they could also entrap proteins or enzymes in vivo. To test this, Cry3Aa* and two copies of PML^{VG} were put under a single cry3Aa promoter with each protein having its own ribosome binding site. As expected, SDS-PAGE analysis demonstrated that PML^{VG} was entrapped in Cry3Aa* crystals with high efficiency and the resulting Cry3Aa*[PML^{VG}]² crystals displayed high activity (FIG. 13).

### Entrapment of PML Inside Cry3Aa-Fusion Crystals

One can envisage that if Cry3Aa-fusion crystals formed pore-like structures they should also be capable of enzyme entrapment. Notably, this would be a facile strategy to produce multi-protein or multi-enzyme immobilized systems. Some advantages to this system over co-entrapment in native Cry3Aa crystals are that the fusion partner will be expressed in a 1:1 ratio as Cry3Aa, so proteins or enzymes that exhibit poor expression levels can be fused to Cry3Aa or Cry3Aa*. The feasibility of this approach by coexpressing Cry3Aa* fused to SpyCatcher (Cry3Aa*-SpyCat) and PML^{VG} to produce Cry3Aa*-SpyCat[PML^{VG}] crystals (FIG. 13a) has already been demonstrated. While SpyCatcher (10 kDa) is not an enzyme, it can act as a reaction handle to conjugate candidate enzymes fused with a Spytag to produce immobilized dual enzyme crystals.

It was recently discovered that CotA-laccase can convert linoleic acid, an abundant fatty acid in waste oils, into azelaic acid, a valuable chemical used in the cosmetic, pharmaceutical and polymer industries.¹⁶ Thus instead of using methanol to produce biodiesel, lipase mediated hydrolysis of the triacylglycerols in water will generate free linoleic acid, which can be subsequently converted to azelaic acid by CotA-laccase (Scheme 4 in FIG. 16B). Since CotA expression is low, it is a good candidate for conjugation to the Cry3Aa*-SpyCat[PML^{VG}] crystals to produce the lipase:lacasse catalyst. Preliminary results described herein indicate successful conjugation of CotA-Spytag Cry3Aa*-SpyCat[PML^{VG}] crystals, with full retention of lacasse activity (FIG. 13).

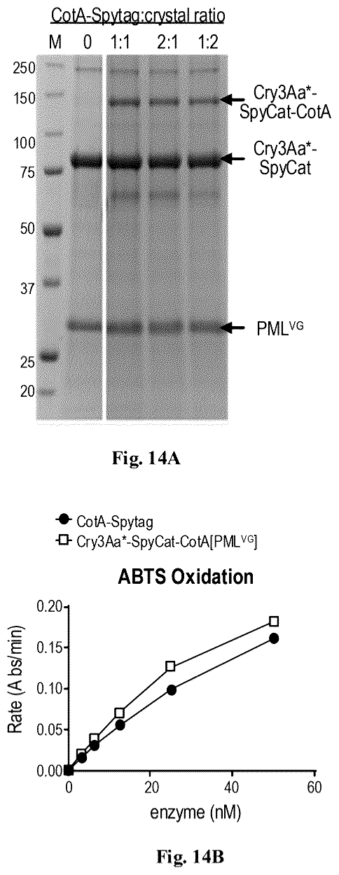

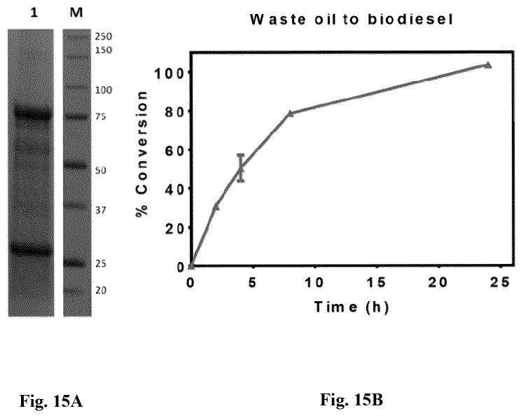

Another example of entrapment of proteins inside Cry3Aa-fusion crystals is the entrapment of PML^{VG} inside Cry3Aa*-lipA crystals. Waste cooking oils are economical feedstocks for biodiesel production, but they have various compositions from source to source. Therefore, production of a single catalyst containing lipases with different chain specificities would be a versatile catalyst for waste cooking oil conversion to biodiesel. The plan was to coexpress Cry3Aa, PML mentioned previously, and lipase A from Bacillus subitilis (lipA) in the same Bt cell. PML prefers long chain fatty acids C₁₄-C₂₀ while lipA prefers medium chain fatty acids C₆-C₁₂. Experimental data showed that PML^{VG} could be entrapped inside Cry3Aa*-lipA crystals and the resulting dual lipase construct was an effective catalyst for waste cooking oil conversion to biodiesel (FIG. 14).

### Example 2

Crystals Formed from Cry3Aa Mutants with Multiple Negatively Charged Amino Acid Substitutions (NegCry3Aa) Enhance the Binding of Certain Proteins

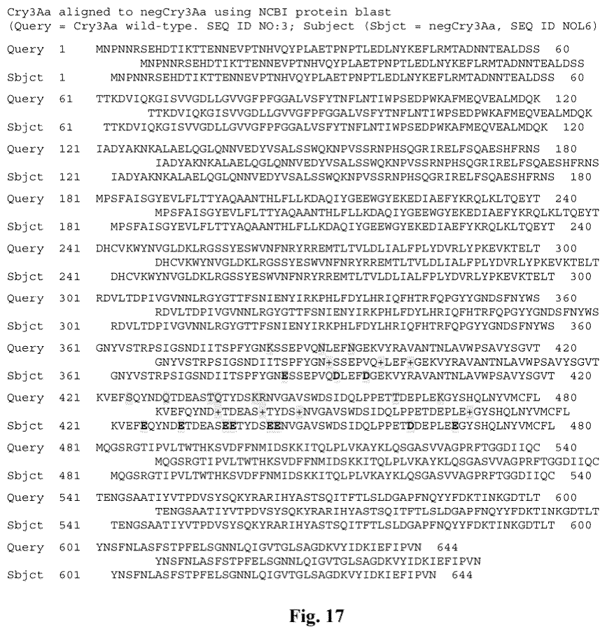

To investigate whether Cry3Aa protein crystal can be engineered to enhance its affinity to the encapsulated protein cargo, a negatively-charged Cry3Aa mutant (negCry3Aa) was generated by substituting specific residues within Domain II that are exposed to the crystal solvent channel with negatively-charged amino acids, namely, aspartate (D) and glutamate (E). For initial studies, the following mutations: K384E, N391D, N395D, S425E, Q430E, TQ436437EE, KR442443EE, T461D, and K467E (bold in the protein sequence alignment of negCry3Aa (SEQ ID NO:6) to Cry3Aa (SEQ ID NO:4) shown in FIG. 17) were engineered into the wild-type cry3aa gene, which was then used to make the final expression construct comprised of the negCry3Aa, a ribosome binding site and the supercharged GFP (scGFP), see Lawrence M S, Phillips K J, Liu D R. Supercharging proteins can impart unusual resilience. J Am Chem Soc. 2007; 129(33):10110-10112. This expression plasmid was then transformed into Bt cells and grown for 48 h or 72 h. Cells were harvested and the resultant crystals (NegCry3Aa-RBS-scGFP) were purified by sucrose density gradient centrifugation, followed by extensive washing in ice-cold ddH₂O. As shown in FIG. 18A, the NegCry3Aa-RBS-scGFP crystal pellets exhibited bright green fluorescence when imaged at the GFP excitation wavelength while no such fluorescence was observed for the control pellet. To further verify the encapsulation of scGFP in negCry3Aa, the negCry3Aa-RBS-scGFP crystal pellets were solubilized in standard SDS sample buffer and boiled for 5 min, followed by SDS-PAGE. As indicated in the SDS-PAGE gel in FIG. 18B, two major bands corresponding to negCry3Aa (theoretical MW=73.1 kDa) and scGFP (theoretical MW=28.5 kDa), respectively, can be observed.

Entrapment of lipAR9 in negCry3Aa Crystals

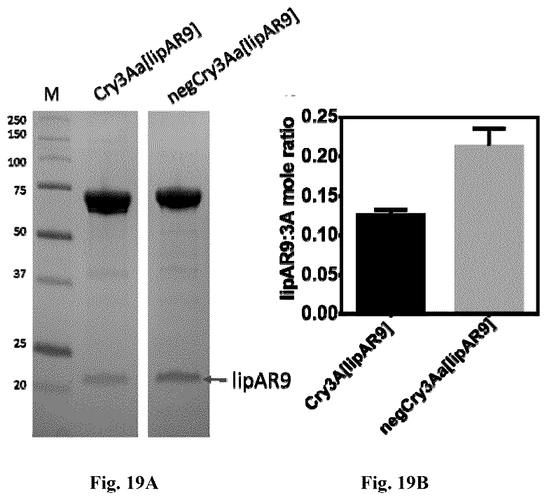

The negCry3Aa mutant also can be used to improve the loading of a lipase. When lipase A fused to polyarginine tail (lipAR9) was co-expressed with negCry3Aa the loading improved (FIG. 19).

### Example 3

### Cry3Aa Fusion Protein Capable of Protein-Entrapping

The present inventors have demonstrated in their previous studies that the fusion of 1-3 repeats of a 56-residue methallothionein protein having the following amino acid sequence (SEQ ID NO:8): MTSTTLVKCACEPCLCNVDPSKAIDRNGLYYCSEACADGHTGGSKGCGHTGCNCHG to the C-terminus of Cry3Aa via genetic fusion can still form fusion crystals (Cry3Aa-[SmtA]₁₋₃) in Bt (Sun, Q., Cheng, SW., Cheung, K., Lee, MM., Chan, MK., “Cry protein crystal-immobilized metallothioneins for bioremediation of heavy metals from water”, Crystals, 2019, 9(6): 287).

The Cry3Aa-[SmtA]₁ construct (a fusion of SEQ ID NO:4 and SEQ ID NO:8) was then tested to reveal whether it can entrap mCherry in vivo when both coding sequences for Cry3Aa-[SmtA]₁ and mCherry are co-expressed in Bt. A pHT315 plasmid harboring the gene encoding sequences of the Cry3Aa-[SmtA]₁, a ribosome binding site, and mCherry was transformed into Bt and grown for 48-72 h. As indicated on the SDS-PAGE gel shown in FIG. 20, the extracts from 40/60% sucrose layer (lane 4) contained the Cry3Aa-[SmtA]₁ (MW 78 kDa) and mCherry (MW ˜26 kDa), thus indicating the entrapment of mCherry in Cry3Aa-[SmtA]₁.

### Example 4

### Changing the Size of Cry3Aa Channel

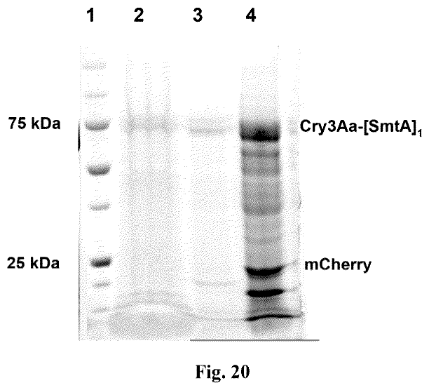

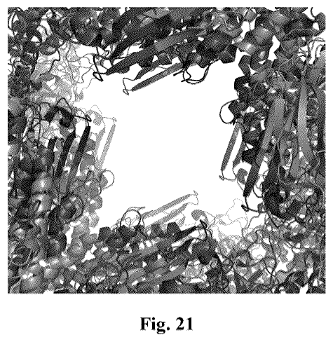

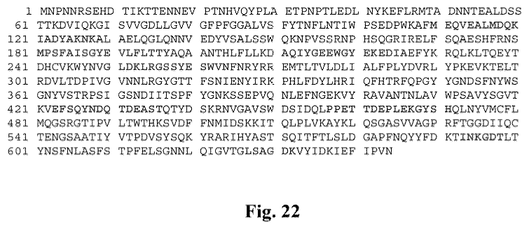

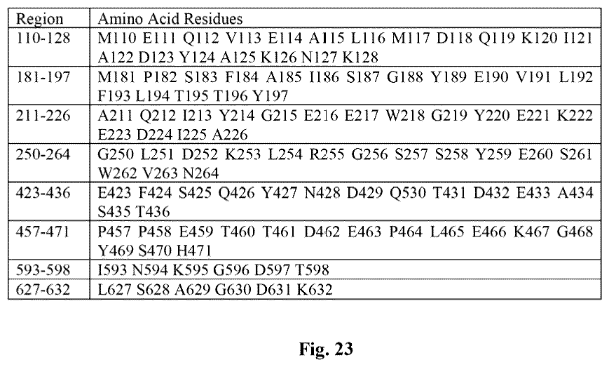

The size of the Cry3Aa solvent channel will influence the size of the protein (e.g., an enzyme) that can be entrapped inside the crystal. For instance, a larger protein may require more space in the channel to be accommodated without steric clash, while a smaller protein might necessitate a smaller channel to prevent its diffusion. In order to increase the size of the channel, amino acids exposed to the solvent channel (FIG. 21) would need to be removed from the Cry3Aa sequence (SEQ ID NO:4). These regions include segments 110-128, 181-197, 211-226, 250-264, 423-436, 457-471, 593-598, and 627-632 of Cry3Aa (SEQ ID NO:4). The specific amino acids that can be deleted are indicated in bold in the Cry3Aa sequence (FIG. 22) and are listed in FIG. 23.

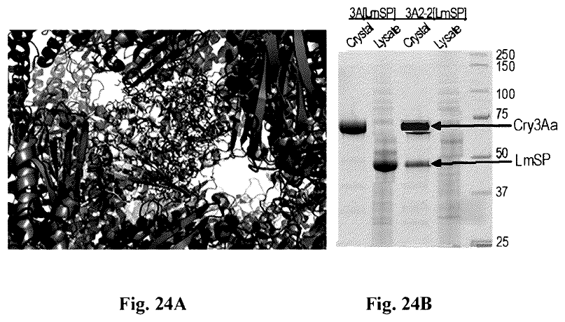

In order to illustrate this for a potential target enzyme, COOT modeling was used to fit a sucrose phosphorylase (LmSP) homolog Thermoanaerobacter thermosaccharolyticum 6F-phosphate phosphorylase (PDB ID: 6S9V) in the Cry3Aa crystal channel. While the majority of LmSP can be accommodated in the crystal, several loops (colored in red) clash with the enzyme model (FIG. 24A). Notably, several of these regions are the same as listed in FIG. 22. One specific area that interferes with LmSP accommodation is the region from 423-436. Therefore, amino acids Gln 426, Tyr 427, Glu 433 and Ala 434 in this region were deleted from Cry3Aa to expand the channel size. When LmSP was co-expressed with this new mutant of Cry3Aa (3A2-2, SEQ ID NO:7), significantly improved entrapment of LmSP in the crystal was observed (FIG. 24B).

Thus, the present inventors have illustrated for the first time that modifying a Cry protein by amino acid insertions or deletions (for example, insertion of at least 1, 2, 3, 4, 5, 6, or 7 amino acid, or a deletion of at least 1, 2, 3, 4, 5, 6, or 7 amino acids) at one or more of the 8 specific regions (for example, at least 1, 2, 3, 4, 5, 6, or 7 regions) shown in FIG. 22 can effectively reduce or enlarge the protein's channel size and can therefore make the resultant crystal-forming protein to entrap a target protein (e.g., an enzyme) with higher level of efficacy, i.e., improved entrapment/retention rate and limited diffusion rate for the target protein.

### Example 5

### Incorporating Mutations in Cry3Aa to Stabilize the Crystal

A very important characteristic of a catalyst is its reusability. Cry3Aa crystals are inherently reusable since they are solid materials. If the crystals solubilize, however, they will release the entrapped enzyme into the bulk media. Chemical cross-linkers can be used to reduce the solubility, but this adds an additional step to catalyst production and poses environmental concerns. Taking advantage of the genetically encoded nature of the Cry3Aa crystal, a more elegant approach is to introduce stabilizing mutations at the intermolecular interfaces to generate an intrinsically insoluble crystal.

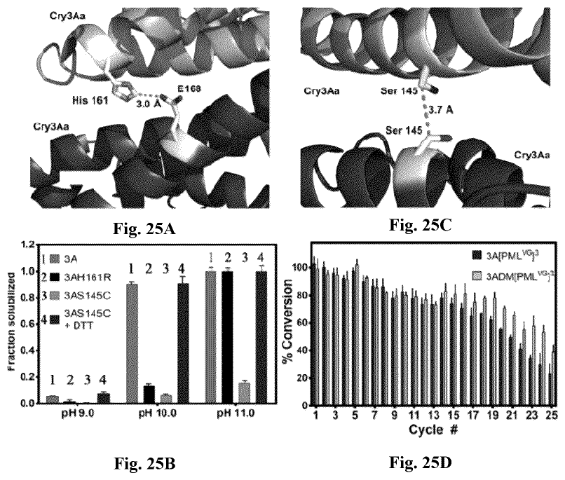

One position where is Ser 145, whose Cu atom is only 3.7 Å away from the Cu of Ser 145 of an adjacent Cry3Aa monomer, making them suitable for disulfide bond formation (FIG. 25a). When Ser 145 was mutated to cysteine (3AS145C) the crystal became much more resistant to solubilization at alkaline pH (FIG. 25b). Solubilization of the crystals in DTT confirmed disulfide bond formation. Another potential region to stabilize is His 161, which interacts with Glu 168 of a neighboring Cry3Aa monomer (FIG. 25c). His 161 was mutated to Arg (3AH161R) to increase the pKa of the charged residue thereby maintaining an electrostatic interaction with Glu 168 across a broader range of pH values. This mutation also reduced the solubility of the Cry3Aa crystal (FIG. 25b). More importantly, when a modified Cry3Aa protein containing both the S145C and H161R mutations was co-expressed with three copies of PML^{VG} (3ADM[PML^{VG}]3), the reusability of the catalyst was enhanced during the production of biodiesel from waste cooking oil (FIG. 25d), increasing the total turnover number (TTN) by 50% after 25 reaction cycles.

### Example 6

### Cry3Aa Crystal Entrapment for Protein Purification

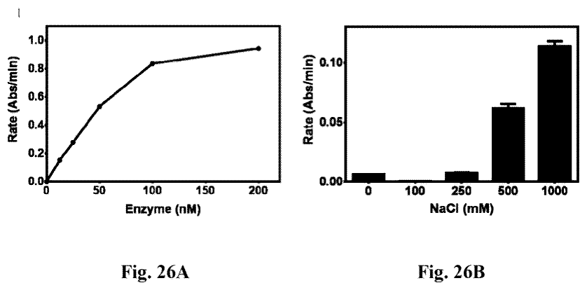

It was previously demonstrated that lysozyme can bind to Cry3Aa crystals in high occupancy, but can be easily released at high salt concentration. It was later demonstrated that the highly positive charge nature of lysozyme was responsible for its binding to Cry3Aa, which has a negatively charge patch of amino acids within its crystal channels. Based on this data, it was speculated that a positively charged patch of amino acids (poly-arginine) can be fused to a protein, coexpress the fusion protein with Cry3Aa in Bacillus thuringiensis, capture the fusion protein in vivo by the Cry3Aa crystal, and release the fusion protein in vitro by adding NaCl. If successful, this can offer anew approach to express and purify soluble protein without the need for columns or expensive reagents. To test this hypothesis, the coding sequence for a poly-arginine tail with 9 consecutive arginine residues (R9) was fused to the coding sequence for lipase A from Bacillus subtilis (lipA, 19 kDa) and inserted downstream of Cry3Aa coding sequence to generate the construct 3A[lipAR9]. As expected, the purified Cry3Aa crystals contained lipAR9 as demonstrated by the high lipase activity of the resulting crystals (FIG. 26A). Notably, lipAR9 can be easily released from the crystal by addition of NaCl (FIG. 26B). These data show that not only can R9 be used as a fusion tag to encapsulate proteins and enzymes within Cry3Aa crystals, but they also confirm that the Cry3Aa platform could be implemented as a general strategy for protein purification.

All patents, patent applications, and other publications, including GenBank Accession Numbers, cited in this application are incorporated by reference in the entirety for all purposes.

1-29. (canceled)

30. An in vitro method of cocrystallizing a Cry protein with at least one protein of interest by mixing soluble Cry protein with the protein of interest, and allowing the Cry protein to form crystal with the protein of interest entrapped within, wherein the Cry protein comprises the amino acid sequence set forth in SEQ ID NO:4, which is optionally modified to comprise:
(i) a truncation of 18-amino acids at the C-terminus;
(ii) substitutions with aspartate (D) or glutamate (E) at residues K384, N391, N395, S425, Q430, T436, Q437, K442, R443, T461, and K467;
(iii) addition of the amino acid sequence set forth in SEQ ID NO:8 in 1-3 repeats at the C-terminus;
(iv) one or more deletions in segment 110-128, 181-197, 211-226, 250-264, 423-436, 457-471, 593-598, or 627-632;
(v) one or more substitutions at residue S145 or H161; or
(vi) the amino acid sequence set forth in SEQ ID NO:5.

31-34. (canceled)

35. The method of claim 30, wherein the at least one protein of interest is an enzyme.

36. The method of claim 35, wherein the enzyme is a lipase, ligase, hydrolase, esterase, protease, or glycosidase.

37. The method of claim 36, wherein the lipase is a Proteus mirabilis lipase (PML), an I118V+E130G mutant of PML (PML^{VG}), a Bacillus subtilis lipase A (lipA), or a lipase A fused to a polyarginine tail (lipAR9).

38. The method of claim 30, wherein the Cry protein comprises the amino acid sequence set forth in SEQ ID NO:4.

39. The method of claim 30, wherein the Cry protein comprises the amino acid sequence set forth in SEQ ID NO:5.

40. The method of claim 30, wherein the Cry protein comprises the amino acid sequence set forth in SEQ ID NO:6.

41. The method of claim 30, wherein the Cry protein comprises the amino acid sequence set forth in SEQ ID NO:7.

42. The method of claim 30, wherein the Cry protein comprises the amino acid sequence set forth in SEQ ID NO:8 fused to the C-terminus of the amino acid sequence set forth in SEQ ID NO:4.

43. The method of claim 30, wherein the Cry protein comprises the amino acid sequence set forth in SEQ ID NO:9.


SEQUENCE LISTING (20 sequences)

SEQ ID NO: 1 - protein, 287 aa, synthetic construct
  REGION 1..287: note = Description of sequence: amino acid sequence of PML
MSTKYPIVLVHGLAGFNEIVGFPYFYGIADALRQDGHQVFTASLSAFNSNEVRGKQLWQFVQTLLQETQAKKVNFIGHSQGPLACRYVAANYPDSVASVTSINGVNHGSEIADLYRRIMRKDSIPEYIVEKVLNAFGTIISTFSGHRGDPQDAIAALESLTTEQVTEFNNKYPQALPKTPGGEGDEIVNGVHYYCFGSYIQGLIAGEKGNLLDPTHAAMRVLNTFFTEKQNDGLVGRSSMRLGKLIKDDYAQDHIDMVNQVAGLVGYNEDIVAIYTQHAKYLASKQL

SEQ ID NO: 2 - protein, 287 aa, synthetic construct
  REGION 1..287: note = Description of sequence: amino acid sequence of PML VG
MSTKYPIVLVHGLAGFNEIVGFPYFYGIADALRQDGHQVFTASLSAFNSNEVRGKQLWQFVQTLLQETQAKKVNFIGHSQGPLACRYVAANYPDSVASVTSINGVNHGSEIADLYRRVMRKDSIPEYIVGKVLNAFGTIISTFSGHRGDPQDAIAALESLTTEQVTEFNNKYPQALPKTPGGEGDEIVNGVHYYCFGSYIQGLIAGEKGNLLDPTHAAMRVLNTFFTEKQNDGLVGRSSMRLGKLIKDDYAQDHIDMVNQVAGLVGYNEDIVAIYTQHAKYLASKQL

SEQ ID NO: 3 - protein, 492 aa, synthetic construct
  REGION 1..492: note = Description of sequence: amino acid sequence of LmSP
MEIQNKAMLITYADSLGKNLKDVHQVLKEDIGDAIGGVHLLPFFPSTGDRGFAPADYTRVDAAFGDWADVEALGEEYYLMFDFMINHISRESVMYQDFKKNHDDSKYKDFFIRWEKFWAKAGENRPTQADVDLIYKRKDKAPTQEITFDDGTTENLWNTFGEEQIDIDVNSAIAKEFIKTTLEDMVKHGANLIRLDAFAYAVKKVDTNDFFVEPEIWDTLNEVREILTPLKAEILPEIHEHYSIPKKINDHGYFTYDFALPMTTLYTLYSGKTNQLAKWLKMSPMKQFTTLDTHDGIGVVDARDILTDDEIDYASEQLYKVGANVKKTYSSASYNNLDIYQINSTYYSALGNDDAAYLLSRVFQVFAPGIPQIYYVGLLAGENDIALLESTKEGRNINRHYYTREEVKSEVKRPVVANLLKLLSWRNESPAFDLAGSITVDTPTDTTIVVTRQDENGQNKAVLTADAANKTFEIVENGQTVMSSDNLTQNLE

SEQ ID NO: 4 - protein, 644 aa, synthetic construct
  REGION 1..644: note = Description of sequence: amino acid sequence of Cry3Aa
MNPNNRSEHDTIKTTENNEVPTNHVQYPLAETPNPTLEDLNYKEFLRMTADNNTEALDSSTTKDVIQKGISVVGDLLGVVGFPFGGALVSFYTNFLNTIWPSEDPWKAFMEQVEALMDQKIADYAKNKALAELQGLQNNVEDYVSALSSWQKNPVSSRNPHSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNKSSEPVQNLEFNGEKVYRAVANTNLAVWPSAVYSGVTKVEFSQYNDQTDEASTQTYDSKRNVGAVSWDSIDQLPPETTDEPLEKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYYFDKTINKGDTLTYNSFNLASFSTPFELSGNNLQIGVTGLSAGDKVYIDKIEFIPVN

SEQ ID NO: 5 - protein, 625 aa, synthetic construct
  REGION 1..625: note = Description of sequence: amino acid sequence of Cry3Aa(1-625) (Cry3Aa*)
MNPNNRSEHDTIKTTENNEVPTNHVQYPLAETPNPTLEDLNYKEFLRMTADNNTEALDSSTTKDVIQKGISVVGDLLGVVGFPFGGALVSFYTNFLNTIWPSEDPWKAFMEQVEALMDQKIADYAKNKALAELQGLQNNVEDYVSALSSWQKNPVSSRNPHSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNKSSEPVQNLEFNGEKVYRAVANTNLAVWPSAVYSGVTKVEFSQYNDQTDEASTQTYDSKRNVGAVSWDSIDQLPPETTDEPLEKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYYFDKTINKGDTLTYNSFNLASFSTPFELSGNNLQIGVT

SEQ ID NO: 6 - protein, 644 aa, synthetic construct
  REGION 1..644: note = Description of sequence: amino acid sequence of negCry3Aa
MNPNNRSEHDTIKTTENNEVPTNHVQYPLAETPNPTLEDLNYKEFLRMTADNNTEALDSSTTKDVIQKGISVVGDLLGVVGFPFGGALVSFYTNFLNTIWPSEDPWKAFMEQVEALMDQKIADYAKNKALAELQGLQNNVEDYVSALSSWQKNPVSSRNPHSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNESSEPVQDLEFDGEKVYRAVANTNLAVWPSAVYSGVTKVEFEQYNDETDEASEETYDSEENVGAVSWDSIDQLPPETDDEPLEEGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYYFDKTINKGDTLTYNSFNLASFSTPFELSGNNLQIGVTGLSAGDKVYIDKIEFIPVN

SEQ ID NO: 7 - protein, 640 aa, synthetic construct
  REGION 1..640: note = Description of sequence: amino acid sequence of 3A2-2
MNPNNRSEHDTIKTTENNEVPTNHVQYPLAETPNPTLEDLNYKEFLRMTADNNTEALDSSTTKDVIQKGISVVGDLLGVVGFPFGGALVSFYTNFLNTIWPSEDPWKAFMEQVEALMDQKIADYAKNKALAELQGLQNNVEDYVSALSSWQKNPVSSRNPHSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNKSSEPVQNLEFNGEKVYRAVANTNLAVWPSAVYSGVTKVEFSNDQTDSTQTYDSKRNVGAVSWDSIDQLPPETTDEPLEKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYYFDKTINKGDTLTYNSFNLASFSTPFELSGNNLQIGVTGLSAGDKVYIDKIEFIPVN

SEQ ID NO: 8 - protein, 56 aa, synthetic construct
  REGION 1..56: note = Description of sequence: amino acid sequence of methallothionein protein
MTSTTLVKCACEPCLCNVDPSKAIDRNGLYYCSEACADGHTGGSKGCGHTGCNCHG

SEQ ID NO: 9 - protein, 644 aa, synthetic construct
  REGION 1..644: note = Description of sequence: amino acid sequence of Cry3Aa double mutant (S145C, H161R)
MNPNNRSEHDTIKTTENNEVPTNHVQYPLAETPNPTLEDLNYKEFLRMTADNNTEALDSSTTKDVIQKGISVVGDLLGVVGFPFGGALVSFYTNFLNTIWPSEDPWKAFMEQVEALMDQKIADYAKNKALAELQGLQNNVEDYVCALSSWQKNPVSSRNPRSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNKSSEPVQNLEFNGEKVYRAVANTNLAVWPSAVYSGVTKVEFSQYNDQTDEASTQTYDSKRNVGAVSWDSIDQLPPETTDEPLEKGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYYFDKTINKGDTLTYNSFNLASFSTPFELSGNNLQIGVTGLSAGDKVYIDKIEFIPVN

SEQ ID NO: 10 - protein, 182 aa, synthetic construct
  REGION 1..182: note = Description of sequence: amino acid sequence of lipA
MAEHNPVVMVHGIGGASFNFAGIKSYLVSQGWSRDKLYAVDFWDKTGTNYNNGPVLSRFVQKVLDETGAKKVDIVAHSMGGANTLYYIKNLDGGNKVANVVTVGGANRLTTGKALPGTDPNQKILYTSIYSSADMIVMNYLSRLDGARNVQIHGVGHIGLLYSSQVNSLIKEGLNGGGQNTN

SEQ ID NO: 11 - protein, 195 aa, synthetic construct
  REGION 1..195: note = Description of sequence: amino acid sequence of lipAR9
MAEHNPVVMVHGIGGASFNFAGIKSYLVSQGWSRDKLYAVDFWDKTGTNYNNGPVLSRFVQKVLDETGAKKVDIVAHSMGGANTLYYIKNLDGGNKVANVVTVGGANRLTTGKALPGTDPNQKILYTSIYSSADMIVMNYLSRLDGARNVQIHGVGHIGLLYSSQVNSLIKEGLNGGGQNTNGSSGRRRRRRRRR

SEQ ID NO: 12 - protein, 633 aa, synthetic construct
  REGION 1..633: note = Description of sequence: consensus sequence from Fig. 17
MNPNNRSEHDTIKTTENNEVPTNHVQYPLAETPNPTLEDLNYKEFLRMTADNNTEALDSSTTKDVIQKGISVVGDLLGVVGFPFGGALVSFYTNFLNTIWPSEDPWKAFMEQVEALMDQKIADYAKNKALAELQGLQNNVEDYVSALSSWQKNPVSSRNPHSQGRIRELFSQAESHFRNSMPSFAISGYEVLFLTTYAQAANTHLFLLKDAQIYGEEWGYEKEDIAEFYKRQLKLTQEYTDHCVKWYNVGLDKLRGSSYESWVNFNRYRREMTLTVLDLIALFPLYDVRLYPKEVKTELTRDVLTDPIVGVNNLRGYGTTFSNIENYIRKPHLFDYLHRIQFHTRFQPGYYGNDSFNYWSGNYVSTRPSIGSNDIITSPFYGNSSEPVQLEFGEKVYRAVANTNLAVWPSAVYSGVTKVEFQYNDTDEASTYDSNVGAVSWDSIDQLPPETDEPLEGYSHQLNYVMCFLMQGSRGTIPVLTWTHKSVDFFNMIDSKKITQLPLVKAYKLQSGASVVAGPRFTGGDIIQCTENGSAATIYVTPDVSYSQKYRARIHYASTSQITFTLSLDGAPFNQYYFDKTINKGDTLTYNSFNLASFSTPFELSGNNLQIGVTGLSAGDKVYIDKIEFIPVN

SEQ ID NO: 13 - protein, 1176 aa, Bacillus thuringiensis
  REGION 1..1176: note = Description of sequence: protein Cry1Aa from Fig. 27
MDNNPNINECIPYNCLSNPEVEVLGGERIETGYTPIDISLSLTQFLLSEFVPGAGFVLGLVDIIWGIFGPSQWDAFPVQIEQLINQRIEEFARNQAISRLEGLSNLYQIYAESFREWEADPTNPALREEMRIQFNDMNSALTTAIPLLAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFSNYDSRRYPIRTVSQLTREIYTNPVLENFDGSFRGMAQRIEQNIRQPHLMDILNSITIYTDVHRGFNYWSGHQITASPVGFSGPEFAFPLFGNAGNAAPPVLVSLTGLGIFRTLSSPLYRRIILGSGPNNQELFVLDGTEFSFASLTTNLPSTIYRQRGTVDSLDVIPPQDNSVPPRAGFSHRLSHVTMLSQAAGAVYTLRAPTFSWQHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKQELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAQSPIGKCGEPNRCAPHLEWNPDLDCSCRDGEKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDQLQADTNIAMIHAADKRVHSIREAYLPELSVIPGVNAAIFEELEGRIFTAFSLYDARNVIKNGDFNNGLSCWNVKGHVDVEEQNNQRSVLVVPEWEAEVSQEVRVCPGRGYILRVTAYKEGYGEGCVTIHEIENNTDELKFSNCVEEEIYPNNTVTCNDYTVNQEEYGGAYTSRNRGYNEAPSVPADYASVYEEKSYTDGRRENPCEFNRGYRDYTPLPVGYVTKELEYFPETDKVWIEIGETEGTFIVDSVELLLMEE

SEQ ID NO: 14 - protein, 633 aa, Bacillus thuringiensis
  REGION 1..633: note = Description of sequence: protein Cry2Aa from Fig. 27
MNNVLNSGRTTICDAYNVVAHDPFSFEHKSLDTIQKEWMEWKRTDHSLYVAPVVGTVSSFLLKKVGSLIGKRILSELWGIIFPSGSTNLMQDILRETEQFLNQRLNTDTLARVNAELIGLQANIREFNQQVDNFLNPTQNPVPLSITSSVNTMQQLFLNRLPQFQIQGYQLLLLPLFAQAANMHLSFIRDVILNADEWGISAATLRTYRDYLRNYTRDYSNYCINTYQTAFRGLNTRLHDMLEFRTYMFLNVFEYVSIWSLFKYQSLMVSSGANLYASGSGPQQTQSFTAQNWPFLYSLFQVNSNYILSGISGTRLSITFPNIGGLPGSTTTHSLNSARVNYSGGVSSGLIGATNLNHNFNCSTVLPPLSTPFVRSWLDSGTDREGVATSTNWQTESFQTTLSLRCGAFSARGNSNYFPDYFIRNISGVPLVIRNEDLTRPLHYNQIRNIESPSGTPGGARAYLVSVHNRKNNIYAANENGTMIHLAPEDYTGFTISPIHATQVNNQTRTFISEKFGNQGDSLRFEQSNTTARYTLRGNGNSYNLYLRVSSIGNSTIRVTINGRVYTVSNVNTTTNNDGVNDNGARFSDINIGNIVASDNTNVTLDINVTLNSGTPFDLMNIMFVPTNLPPLY

SEQ ID NO: 15 - protein, 1180 aa, Bacillus thuringiensis
  REGION 1..1180: note = Description of sequence: protein Cry4Aa from Fig. 27
MNPYQNKNEYETLNASQKKLNISNNYTRYPIENSPKQLLQSTNYKDWLNMCQQNQQYGGDFETFIDSGELSAYTIVVGTVLTGFGFTTPLGLALIGFGTLIPVLFPAQDQSNTWSDFITQTKNIIKKEIASTYISNANKILNRSFNVISTYHNHLKTWENNPNPQNTQDVRTQIQLVHYHFQNVIPELVNSCPPNPSDCDYYNILVLSSYAQAANLHLTVLNQAVKFEAYLKNNRQFDYLEPLPTAIDYYPVLTKAIEDYTNYCVTTYKKGLNLIKTTPDSNLDGNINWNTYNTYRTKMTTAVLDLVALFPNYDVGKYPIGVQSELTREIYQVLNFEESPYKYYDFQYQEDSLTRRPHLFTWLDSLNFYEKAQTTPNNFFTSHYNMFHYTLDNISQKSSVFGNHNVTDKLKSLGLATNIYIFLLNVISLDNKYLNDYNNISKMDFFITNGTRLLEKELTAGSGQITYDVNKNIFGLPILKRRENQGNPTLFPTYDNYSHILSFIKSLSIPATYKTQVYTFAWTHSSVDPKNTIYTHLTTQIPAVKANSLGTASKVVQGPGHTGGDLIDFKDHFKITCQHSNFQQSYFIRIRYASNGSANTRAVINLSIPGVAELGMALNPTFSGTDYTNLKYKDFQYLEFSNEVKFAPNQNISLVFNRSDVYTNTTVLIDKIEFLPITRSIREDREKQKLETVQQIINTFYANPIKNTLQSELTDYDIDQAANLVECISEELYPKEKMLLLDEVKNAKQLSQSRNVLQNGDFESATLGWTTSDNITIQEDDPIFKGHYLHMSGARDIDGTIFPTYIFQKIDESKLKPYTRYLVRGFVGSSKDVELVVSRYGEEIDAIMNVPADLNYLYPSTFDCEGSNRCETSAVPANIGNTSDMLYSCQYDTGKKHVVCQDSHQFSFTIDTGALDTNENIGVWVMFKISSPDGYASLDNLEVIEEGPIDGEALSRVKHMEKKWNDQMEAKRSETQQAYDVAKQAIDALFTNVQDEALQFDTTLAQIQYAEYLVQSIPYVYNDWLSDVPGMNYDIYVELDARVAQARYLYDTRNIIKNGDFTQGVMGWHVTGNADVQQIDGVSVLVLSNWSAGVSQNVHLQHNHGYVLRVIAKKEGPGNGYVTLMDCEENQEKLTFTSCEEGYITKTVDVFPDTDRVRIEIGETEGSFYIESIELICMNE

SEQ ID NO: 16 - protein, 1245 aa, Bacillus thuringiensis
  REGION 1..1245: note = Description of sequence: protein Cry5B from Fig. 27
MATINELYPVPYNVLAHPIKEVDDPYSWSNLLKGIQEGWEEWGKTGQKKLFEDHLTIAWNLYKTGKLDYFALTKASISLIGFIPGAEAAVPFINMFVDFVWPKLFGANTEGKDQQLFNAIMDAVNKMVDNKFLRYNLSTLNKTIEGLQGNLGLFQNPIQVAICQGSTPERVIFDQNCTPCNPNQPCKDDLDRVASRFDTANSQFTQHLPEFKNPWSDENSTQEFKRTSVELTLPLYTTVATLHLLLYKGYIEFMTKWNFHNAQYLNNLKVELQQLIHSYSETVRTSFLQFLPTLNNRSKSSVNAYNGYVRNMTVNCLDIAATWPTFDTHNYHQGRKLDLTRIILSDTAGPIKEYTTGDKTSGPQHSNITPNNILDTPSPTYQHSFVSVDSIVYSRKELQQLDIATYSTNNSNNCHPYGLRLSYTDGSRYDYGDNQPDFTTSNNNYCHNSYTAPITLVNARHLYNAKGSLQNVESLVVSTVNGGSGSCICDAWINYLRPPQTSKNESRPDQKINVLYPITETVNKGTGGNLGVISAYVPMELVPENVIGDVNADTKLPLTQLKGFPFEKYGSEYNNRGISLVREWINGNNAVKLSNSQSVGIQITNQTKQKYEIRCRYASKGDNNVYFNVDLSENPFRNSISFGSTESSVVGVQGENGKYILKSITTVEIPAGSFYVHITNQGSSDLFLDRIEFVPKIQFQFCDNNNLHCDCNNPVDTDCTFCCVCTSLTDCDCNNPRGLDCTLCCQVENQLPSFVTLTDLQNITTQVNALVASSEHDTLATDVSDYEIEEVVLKVDALSGEVFGKEKKALRKLVNHTKRLSKARNLLIGGNFDNLDAWYRGRNVVNVSDHELFKSDHVLLPPPTLYSSYMFQKVEESKLKANTRYTVSGFIAHAEDLEIVVSRYGQEVKKVVQVPYGEAFPLTSRGAICCPPRSTCNGKPADPHFFSYSIDVGTLDVEANPGIELGLRIVERTGMARVSNLEIREDRPLKKNELRNVQRAARNWRTAYDQERAEVTALIQPVLNQINALYENEDWNRAIRSGVSYHDLEAIVLPTLPKLNHWFMSDMLGEQGSILAQFQEALDRAYTQLEESTILHNGHFTTDAANWTIEGDAHHAILEDGRRVLRLPDWSSSVSQTIEIENFDPDKEYQLVFHAQGEGTVSLQHGEEGEYVETHPHKSANFTTSHRQGVTFETNKVTVEITSEDGEFLVDHIALVEAPLPTDDQSSDGNTTSNTNSNTSMNNNQ

SEQ ID NO: 17 - protein, 609 aa, Bacillus thuringiensis
  REGION 1..609: note = Description of sequence: protein Cry7Ca1 from Fig. 27
MRGSHHHHHHGSMIFSSISIIRTFMGFAGHGTAGGIIGLFTEVLRLLWPNKQNDLWESFMNEVEALINQEITEAVVSKALSELEGLRNALEGYTSALEAWQNNRSDKLKQLLVYERFVSTENLFKFAMPSFRSVGFEGPLLTVYAQAANLHLFLLKNAELFGAEWGMQQYEIDLFYNEQKGYVEEYTDHCVKWYKEGLNKLKNASGVKGKVWENYNRFRREMTIMVLDLLPLFPIYDARTYPMETVTELTRQIFTDPIGLTGINETKYPDWYGAASSEFVLIENRAIPKPGLFQWLTKINVRARVVEPNDRFAIWTGHSVVTQYTKSTTENTFNYGTSSGSTLSHTFDLLSKDIYQTYSIAAANKSATWYQAVPLLRLYGINSSNVLSEDAFSFSNNIPSSKCKSTYSSDQLPIELLDEPIYGDLEEYGHRLSYVSEIFKETGSGTIPVLGWTHVSVRPDNKLYPDKITQIPAVKAFETNTAGVEIIDSASTGGPILKIVNNNLPSNQVFRMRLSFSEPQKIKVRVRYAATGDGVMSFSGIAHDEYFTATMKEGEALKYSYLTMGNDYAGTAAELSMLYIIKANTSNCTIYIDKIEFIPVVDLQPSLIS

SEQ ID NO: 18 - protein, 1164 aa, Bacillus thuringiensis
  REGION 1..1164: note = Description of sequence: protein Cry8Ea1 from Fig. 27
MSPNNQNEYEIIDMAPSTSVSNDSNRYPFASDPTNALQNMNYKEYLRMSEGYDSEYSGSPEVLISERDAVKTAISLVGTILGKLGVPLVGPIVSLYSTLIDVLWPGGKSQWEIFMEQVEALINQKIAEYARAKALAELEGLGNNYQLYLTALEEWQENPSSTRVLRDVRNRFEILDSLFTQYMPSFRVTGYEVPLLSVYAQAANLHLLLLKDASIFGEEWGFSTTAINNYYNRQMSLIAQYSDHCVQWYRTGLDRLKGSNAKQWVEYNRFRREMTLSVLDIMTLFPMYDMRTYPMETKAQLTREVYTDPIGAIGAQGSWYDSAPSFNTLESTFIRGKHLFDFITRLSIYTGRSSFSASNYLKKWIGHQISSQPIGGSIQTQTYGTTSGSSVIATQQIGFTGFDVYKTLSTAGVLFAYTSKYYGVSKVVFDAIYPDNKYKTTFTYNPGSEGIGAQEKDSEVELPPETLDQPNYEAYSHRLNYVTFIRNPDVPVFSWTHRSADRTNTVYSDKITQIPVVKASDGPKPSANEVGHYLGGDPISFNSSGSTGVIRLNINSPLSQKYRVRIRYCSSVDFDLDVVRGGTTVNNGRFNKSAPNVGWQSLKYENFKFASFSTPFTFNQAQDTLKISVRNFSSIVGGSVVYIDRIELIPVNATYEAEQDLDSAKKAVNTLFTNTKDGLRPGVTDYEVNQAANLVECLSDDLYPNEKRLLFDAVKEAKRLSEARNLLQDPDFQEINGENGWTASTGIEVVEGDALFKGRYLRLPGAREMDTETYPTYLYQKVEEGVLKPYTRYRLRGFVGSSQGLEISTIRHQTNRIVKNVPDDLLPDVPPVNSDGRINRCSEQKYVNSRLEGERGLPNGNRSAEAHEFSLPIDIGELDYNENAGIWVGFKITDPEGYATLGNLELVEEGPLSGDALERLQREEQQWKLQMTKRREETDRKYTAAKQAVDRLYADYQDQQLNPNVEITDITAAQNLIQSIPYVYNEMFPEIQGMNYTKYTELTNRLQQAWGLYDQRNAIPNGDFRNELSNWNTTSGVNVQQINNTSVLVMPNWDGQVSQQFTVQPNQRYVLRVTARKEGVGNGYVSIRDGGNQTETLTFSASDYNTDSVYNTQVSNTNGLYNEQTGYTTKTVTFIPYTDQVWIEMSETEGMFYIESVELIVDVE

SEQ ID NO: 19 - protein, 675 aa, Bacillus thuringiensis
  REGION 1..675: note = Description of sequence: protein Cry10Aa from Fig. 27
MNPYQNKNEYEIFNAPSNGFSKSNNYSRYPLANKPNQPLKNTNYKDWLNVCQDNQQYGNNAGNFASSETIVGVSAGIIVVGTMLGAFAAPVLAAGIISFGTLLPIFWQGSDPANVWQDLLNIGGRPIQEIDKNIINVLTSIVTPIKNQLDKYQEFFDKWEPARTHANAKAVHDLFTTLEPIIDKDLDMLKNNASYRIPTLPAYAQIATWHLNLLKHAATYYNIWLQNQGINPSTFNSSNYYQGYLKRKIQEYTDYCIQTYNAGLTMIRTNTNATWNMYNTYRLEMTLTVLDLIAIFPNYDPEKYPIGVKSELIREVYTNVNSDTFRTITELENGLTRNPTLFTWINQGRFYTRNSRDILDPYDIFSFTGNQMAFTHTNDDRNIIWGAVHGNIISQDTSKVFPFYRNKPIDKVEIVRHREYSDIIYEMIFFSNSSEVFRYSSNSTIENNYKRTDSYMIPKQTWKNEEYGHTLSYIKTDNYIFSVVRERRRVAFSWTHTSVDFQNTIDLDNITQIHALKALKVSSDSKIVKGPGHTGGDLVILKDSMDFRVRFLKNVSRQYQVRIRYATNAPKTTVFLTGIDTISVELPSTTSRQNPNATDLTYADFGYVTFPRTVPNKTFEGEDTLLMTLYGTPNHSYNIYIDKIEFIPITQSVLDYTEKQNIEKTQKIVNDLFVN

SEQ ID NO: 20 - protein, 643 aa, Bacillus thuringiensis
  REGION 1..643: note = Description of sequence: protein Cry11Aa from Fig. 27
MEDSSLDTLSIVNETDFPLYNNYTEPTIAPALIAVAPIAQYLATAIGKWAAKAAFSKVLSLIFPGSQPATMEKVRTEVETLINQKLSQDRVNILNAEYRGIIEVSDVFDAYIKQPGFTPATAKGYFLNLSGAIIQRLPQFEVQTYEGVSIALFTQMCTLHLTLLKDGILAGSAWGFTQADVDSFIKLFNQKVLDYRTRLMRMYTEEFGRLCKVSLKDGLTFRNMCNLYVFPFAEAWSLMRYEGLKLQSSLSLWDYVGVSIPVNYNEWGGLVYKLLMGEVNQRLTTVKFNYSFTNEPADIPARENIRGVHPIYDPSSGLTGWIGNGRTNNFNFADNNGNEIMEVRTQTFYQNPNNEPIAPRDIINQILTAPAPADLFFKNADINVKFTQWFQSTLYGWNIKLGTQTVLSSRTGTIPPNYLAYDGYYIRAISACPRGVSLAYNHDLTTLTYNRIEYDSPTTENIIVGFAPDNTKDFYSKKSHYLSETNDSYVIPALQFAEVSDRSFLEDTPDQATDGSIKFARTFISNEAKYSIRLNTGFNTATRYKLIIRVRVPYRLPAGIRVQSQNSGNNRMLGSFTANANPEWVDFVTDAFTFNDLGITTSSTNALFSISSDSLNSGEEWYLSQLFLVKESAFTTQINPLLK